(19) United States
(12) Patent Application Publication
(10) Pub. No.: US 2016/0376321 A1
(43) Pub. Date: Dec. 29, 2016

(54) A NOVEL SARS IMMUNOGENIC COMPOSITION

(71) Applicant: BAYLOR COLLEGE OF MEDICINE, Houston, TX (US)

(72) Inventors: Peter Jay Hotez, Houston, TX (US); Maria Elena Bottazzi, Houston, TX (US); Bin Zhan, Pearland, TX (US); Wen-Hsiang Chen, Houston, TX (US); Shivali Chag, Houston, TX (US)

(73) Assignee: BAYLOR COLLEGE OF MEDICINE, Houston, TX (US)

(21) Appl. No.: 15/038,726

(22) PCT Filed: Nov. 21, 2014
(86) PCT No.: PCT/US14/66843

§ 371 Date: May 23, 2016

Related U.S. Application Data
(60) Provisional application No. 61/909,145, filed on Nov. 26, 2013.

(51) Int. Cl. C07K 14/005

(52) U.S. Cl. CPC C07K 14/005; C12N 2760/18522; A61K 38/00

(57) ABSTRACT

Embodiments of the disclosure concern immunogenic compositions and methods for treating or preventing Severe acute respiratory syndrome (SARS). The compositions and methods concern a portion of the receptor-binding domain (RBD) of the SARS-CoV spike protein. In at least particular cases, a mutated version of a portion of the RBD is utilized, such as a deglycosylated mutant of the RBD.

This application claims priority to U.S. Provisional Patent Application Ser. No. 61/909,145, filed Nov. 26, 2013, which is incorporated by reference herein in its entirety.

### STATEMENT REGARDING FEDERALLY SPONSORED RESEARCH OR DEVELOPMENT

This invention was made with government support under R01AI098775 awarded by the National Institutes of Health. The government has certain rights in the invention.

### TECHNICAL FIELD

The field of the disclosure concerns at least the fields of cell biology, molecular biology, immunology, virology, biochemistry, vaccinology, and medicine.

### BACKGROUND

Severe acute respiratory syndrome (SARS) emerged in Guangdong Province in South China in 2002, ultimately spreading to five continents where it caused 8,000 respiratory infections and 800 deaths (Du et al., 2009). The SARS coronavirus (SARS-CoV) was identified as the etiologic agent of SARS in 2003 (Peiris et al., 2003; Zhong et al., 2003) and subsequently defined by the National Institute of Allergy and Infectious Diseases (NIAID) of the U.S. National Institutes of Health (NIH) as a Category C pathogen, along with other highly transmissible agents of potential biodefense importance (Jiang et al., 2012).

Because of the explosive nature of the 2002-03 SARS pandemic, an intensive effort has been underway to develop SARS countermeasures, including vaccines (Du et al., 2009). A stable and effective SARS-CoV vaccine could be stockpiled as part of national or global public health emergency preparedness efforts (Jiang et al., 2012). Initial efforts focused on developing whole virus vaccines that were often inactivated by chemical agents or radiation and adjuvanted on alum (Du et al., 2009). However, in laboratory mice, it was observed that such vaccines elicited cosinophilic immunoenhancing pathology with evidence of Th2-linked alveolar damage (Perlman et al., 2005; Balles et al., 2011). Previously, immune enhancing pathology in vaccinated children derailed similar efforts to develop inactivated respiratory syncytial virus (RSV) vaccines (Castilow et al., 2007).

As an alternative approach, prototype subunit vaccines comprised of the SARS-CoV spike (S) protein have been developed (Du et al., 2009). Like HIV gp160 and influenza hemagglutinin, the SARS-CoV S protein is a class I viral fusion protein, and, as such, it is a major target of host neutralizing antibodies (Du et al., 2009; Jiang et al., 2012). Efforts to develop genetically engineered SARS-CoV S protein vaccines were reviewed previously (Du et al., 2009). Briefly, both baculovirus-expressed recombinant protein adjuvanted with alum and a Venezuelan equine encephalitis vector containing S-protein plasmid were shown to elicit protection in BALB/c mice challenged with live SARS-CoV (Du et al., 2009; Tseng et al., 2012), but some S-protein constructs expressed in mammalian cells were found to cause antibody-mediated enhancement (Jaume et al., 2012).

As a substitute for the full-length S protein, its 193 amino acids (aa) minimal receptor-binding domain (RBD) containing residues 318-510 (RBD193) was identified and found to bind to its putative human receptor, a transmembrane angiotensin-converting enzyme 2 (ACE2), in vitro (Wong et al., 2004). In addition, recombinant proteins RBD193 and a related construct, RBD219 (residues 318-536), expressed in the culture supernatant of mammalian cells 293T and Chinese hamster ovary (CHO)-K1, respectively, were demonstrated to elicit neutralizing antibodies and protective immunity in vaccinated mice (Du et al., 2009; Du et al., 2012). Moreover, RBD can also absorb and remove the majority of neutralizing antibodies in the antisera of mice, monkeys, and rabbits immunized with whole SARS-CoV or vaccinia virus expressing S protein constructs (Chen et al., 2005).

The present disclosure provides a solution to a long-felt need in the art by providing a SARS immunogenic composition that does not elicit eosinophilic immunopathology or antibody-mediated enhancement of disease and does not cause harmful immune responses, while at the same time inducing a potent cross-neutralizing antibody response compared to other SARS vaccines.

### BRIEF SUMMARY

Embodiments of the disclosure concern methods and/or compositions related to Severe Acute Respiratory Syndrome (SARS) treatment or prevention, including complete prevention or reduction in severity of one or more symptoms or delay in onset of one or more symptoms of SARS, for example. In particular aspects, there are methods and/or compositions related to the SARS-CoV spike protein useful for treatment or prevention of SARS. In certain embodiments, the receptor-binding domain (RBD) of the SARS-CoV spike protein (the RBD is from the subunit 1(S1) of spike protein) is related to SARS treatment or prevention. In specific embodiments, there are methods and/or compositions concerning one or more modified RBDs of the SARS CoV spike protein for treatment or prevention of SARS. In particular cases, the RBD modification comprises deletion and/or mutation of one or more glycosylation sites of the RBD sequence. In certain aspects, the RBD-modified composition lacks one or more asparagine-linked glycosylation sites, such as by removal of the first asparagine (RBD219-N1, RBD193-N1) (such as by substitution or physical removal) or by substitution or removal of one or both of the two remaining asparagines, and in some cases in addition to the deletion of the first asparagine (RBD219-N3, RBD193-N3), for example. In some cases, modified RBD compositions may have amino acid substitutions, deletions, inversion, and so forth. In particular embodiments, the modified RBD composition has a modification other than at a glycosylation site. Some embodiments include RBDs that are modified to include deletion of an amino acid that is glycosylated under normal conditions. Some aspects of the disclosure concern RBDs that are modified to include substitutions of an asparagine to another amino acid (such as serine or aspartate, for example). Certain cases include modifications at more than one amino acid in a given RBD protein molecule, including at more than one asparagine, for example. In specific embodiments, the composition is isolated, recombinant, synthetic, and/or not found in nature.

In specific embodiments, one or more immunogenic compositions and/or methods are employed in an individual for the prevention of SARS or delay in onset of SARS and/or reduction of severity of at least one symptom of SARS.

Embodiments of the disclosure include development of a SARS immunogenic composition, such as a vaccine, comprising a receptor-binding domain of the SARS-CoV spike protein. The vaccine or immunogenic composition may comprise one or more adjuvants. In particular cases, the vaccine or immunogenic composition may be expressed as recombinant protein in yeast or in a mammalian system, for example.

In embodiments of the invention, yeast-expressed recombinant protein of the receptor-binding domain in SARS-CoV spike protein with deglycosylation is useful as a SARS immunogenic composition or vaccine.

In particular cases, there are RBD193 and RBD219 recombinant proteins, and expression thereof, as well as their deglycosylated forms. In specific embodiments, the recombinant proteins are produced in the exemplary system of the yeast Pichia pastoris. One of the mutants in particular, RBD219-N1, in which an N-linked glycosylated asparagine at the N-1 position of RBD219 had been deleted, may be expressed as recombinant protein and purified in high yield, and maintain its functionality and antigenicity similar to or even better than the mammalian-expressed RBD193. RBD219-N1 (such as in combination with an alum-based adjuvant) elicited high titers of neutralizing antibodies against both SARS-CoV pseudovirus and live virus. Therefore, this molecule is useful for scale-up process development and manufacture as a recombinant SARS immunogenic composition, such as a vaccine, for example.

In particular aspects of the invention, the infection being addressed by methods and/or compositions of the disclosure is infection by a SARS- or SAES-related virus, such as a genetically related virus for Middle East Respiratory Syndrome (MERS). In some cases, the infection is a coronavirus that may or may not be SARS.

In specific embodiments, one or more immunogenic compositions and/or methods of the disclosure are employed in an individual for the treatment or prevention of MERS or delay in onset of MERS and/or reduction of severity of at least one symptom of MERS.

In some cases, an individual is of any age who is possibly exposed to SARS or a SARS-related infection or a SARS or SARS-related bioweapon, including a child, an elderly person, a member of the military, or a health care worker, for example. The individual may be in or may have been present in a geographical area known to have individuals with SARS or prone to having individuals with SARS.

In embodiments of the disclosure, there is an isolated composition comprising the receptor-binding domain (RBD) of the Severe acute respiratory syndrome coronavirus (SARS-CoV) protein, wherein said domain lacks at least one glycosylation site or is deglycosylated at least at one site that is glycosylated under normal conditions. In some cases, the domain is comprised within the full-length SARS CoV spike protein, whereas in other cases, the domain is a fragment of the SARS-CoV spike protein. In specific embodiments, the fragment comprises amino acid residues 275-575, 300-550, 310-525, or 318-510 of the SARS CoV spike protein or the fragment comprises amino acid residues 275-575, 300-550, 310-540, or 318-536 of the SARS CoV spike protein. In specific embodiments, the fragment is at least 190 amino acids in length or the fragment is at least 210 amino acids in length.

In particular aspects of the disclosure, the glycosylation site is an N-glycosylation site. The N-glycosylation site may be an asparagine site. The site may be at the asparagine at amino acid 318 of the SARS-CoV spike protein, at the asparagine at amino acid 330 of the SARS-CoV spike protein, or at the asparagine at amino acid 347 of the SARS-CoV spike protein. In specific embodiments, the site is at one or more asparagines selected from the group consisting of amino acid 318, amino acid 330, or amino acid 347 of the SARS-CoV spike protein. In particular cases, the fragment comprises amino acid residues 318-536 of the SARS CoV spike protein and the site is at the asparagine at amino acid 318 of the SARS-CoV spike protein. In some cases, the site comprises an amino acid deletion or an amino acid substitution, such as to a serine or alanine.

Any composition of the disclosure may be comprised in a pharmaceutically acceptable vehicle.

In one embodiment, there is a method of preventing or delaying the onset of SARS or of reducing at least one symptom of SARS in an individual, comprising the step of providing an effective amount of any of the compositions of the disclosure to the individual. In particular aspects, a composition is provided to the individual once or more than once. The composition may be provided subsequently to the individual within weeks, months, or years of the first providing step. In some cases, an individual displays one or more symptoms of SARS, lacks any symptoms of SARS, or has been exposed to SARS. In certain aspects, the individual has come into contact with an individual that has SARS. In particular facets, the individual is a child, an elderly person, exposed to a bioweapon or at risk thereof, or is a health care worker.

In one embodiment, there is a method of preventing or delaying the onset of MERS or of reducing at least one symptom of MERS in an individual, comprising the step of providing an effective amount of any of the compositions of the disclosure to the individual. In particular aspects, a composition is provided to the individual once or more than once. The composition may be provided subsequently to the individual within weeks, months, or years of the first providing step. In some cases, an individual displays one or more symptoms of MERS, lacks any symptoms of MERS, or has been exposed to MERS. In certain aspects, the individual has come into contact with an individual that has MERS. In particular facets, the individual is a child, an elderly person, exposed to a bioweapon or at risk thereof, or is a health care worker.

### BRIEF DESCRIPTION OF THE DRAWINGS

FIG. 1. Schematic diagram of the different SAR-CoV S-RBD protein expression constructs. Both RBD193-WT and RBD219-WT contain 3 N-glycosylation sites, including N-1, N-13, and N-40. The deleted or mutated N-glycosylation sites are highlighted with a line crossing the amino acid (e.g., N-1) or italics (e.g., S-13, and A-40), respectively.

FIG. 2. Expression profiles of the different SAR-CoV RBD protein constructs in yeast. The expression of wild-type (WT) and the different deglycosylated proteins of RBD193 and RBD219 in P. pastoris X-33 after induction with methanol were detected by: (A) SDS-PAGE and by (B) Western blot with anti-RBD mAb 33G4 (0.2 μg/ml). Each lane was loaded with 10 μl of induced culture (unpurified). (C) The N-linked glycan on yeast-expressed recombinant RBD193-WT could be removed completely by Peptide-N-Glycosidase F (PNGase F) digestion. Lane 1: protein marker, lane 2: RBD193-expressed culture (10 μl), lane 3: PNGase F digested RBD193.

FIG. 3. Detection of the expression of RBD193-WT protein in yeast by Western blot. RBD193-WT was induced in P. pastoris culture with different pH (Lane 1: markers; Lane 2: pH 5.2; Lane 3: pH 6.0; Lane 4: pH 7.5; and Lane 5: pH 8.0) and with different amounts of detergent (Lane 6: pH 6.0 w/0.01% Empigen and lane 7: pH 6.0 w/0.05% Empigen). The expressed proteins in the medium were transferred on PVDF and probed with 0.2 μg/ml of anti-RBD mAb 33G4.

FIG. 4. SDS-PAGE and Western blot analysis of yeast-expressed RBD proteins. SDS-PAGE (SDS, left panels) and Western blot (WB, right panels) analysis of 2 μg purified RBD 193-N1(A), RBD193-N3 (B), RBD219-WT (C) and RBD219-N1 (D) were performed. Western blot was probed with 0.2 g/ml of anti-RBD mAb 33G4.

FIG. 5. Antigenicity of yeast-expressed RBD proteins. To detect antigenicity mAbs specific for the conformational epitopes of SARS-CoV RBD and Western blot were used. The mAbs 35B5 (Conf IV), 33G4 (Conf V), 24H8 (Conf I), and 31H12 (Conf II) at 0.2 μg/ml were used for the test. Protein molecular weight marker (Marker) was indicated on the left.

FIG. 6. Detection of the reactivity of yeast-expressed RBD proteins with RBD-specific mAbs by ELISA. The mAbs specific for the conformational epitopes (24H8 (Conf I), 19B2 (VI), 35B5 (Conf IV), 33G4 (Conf V), 31H12 (Conf II)), and linear epitopes (17H9) of SARS-CoV RBD were tested at 2.2 μg/ml (A) or 0.25 μg/ml (B), respectively. Wild-type SARS-CoV RBD protein expressed in 293T cells (RBD193-WT) (Du et al., 2009) was included as the positive control. The data in (A) and (B) are expressed as the mean±standard deviation (SD) of duplicate wells.

FIG. 7. Binding of SARS-CoV RBD proteins with cell-associated ACE2 (ACE2/293T cells) or sACE2. Binding of RBD proteins (20 μg/each) with ACE2/293T cells (A) or sACE2 (20 μg) (B-C) was detected by Western blot using goat anti-ACE2 mAb (0.2 μg/ml) or anti-RBD of SARS-CoV mAb (33G4, 1 μg/ml). A recombinant protein containing RBD of Middle East respiratory syndrome coronavirus (MERS-CoV) (Du et al., 2011) was included as the negative control (control).

FIG. 8. Detection of SARS-CoV RBD-specific IgG antibody by ELISA in the vaccinated mouse sera. Sera collected at 10 days post-last vaccination were used for the test. Alum adjuvant plus PBS was used as the control. The data are presented as geometric mean titers (GMT) of five mice per group. P values indicate significant differences between different vaccination groups.

FIG. 9. Detection of neutralizing antibodies in sera of vaccinated mice against pseudotyped and live SARS-CoV infection. Sera collected at 10 days post-last vaccination were used for the test. Alum adjuvant plus PBS was used as the control. (A) Titers of neutralizing antibodies against SARS pseudovirus. The data are expressed as 50% neutralizing antibody titer (NT50) and are presented as mean±SD of five mice per group. (B) Titers of neutralizing antibodies against live SARS-CoV infection. The neutralizing antibody titers were expressed as the reciprocal of the highest dilution of serum that completely prevented virus-induced CPE in at least 50% of the wells (NT50) and presented as mean±SD of five mice per group. P values indicate significant differences between different vaccination groups.

### DETAILED DESCRIPTION

In keeping with long-standing patent law convention, the words “a” and “an” when used in the present specification in concert with the word comprising, including the claims, denote “one or more.” Some embodiments may consist of or consist essentially of one or more elements, method steps, and/or methods of the disclosure. It is contemplated that any method or composition described herein can be implemented with respect to any other method or composition described herein embodiments which are disclosed and still obtain a like or similar result without departing from the spirit and scope of the subject matter.

The term “effective amount” as used herein is defined as the amount of a compound required to prevent SARS or SARS-related infection or delay the onset of or improve at least one symptom of SARS or a SARS-related disease. For example, in the treatment or prevention of SARS or a SARS-related disease, a compound that improves inhibits development of at least one symptom or delays the onset or reduces the severity of at least one symptom would be effective. In embodiments, an effective amount of a compound is not required to cure a disease but will provide a treatment or prevention for a disease.

### I. General Embodiments

Severe acute respiratory syndrome (SARS) emerged in China in 2002 and ultimately caused 8,000 infections and 800 deaths worldwide. It is a Category C pathogen, thus development of a vaccine for preventing a future pandemic and for biodefense preparedness is needed. Previous studies have shown that a SARS vaccine candidate consisting of the receptor-binding domain (RBD) of the SARS-CoV spike protein can induce potent neutralizing antibody responses and protection against a SARS-CoV challenge in vaccinated animals. However, the expression of recombinant RBD in previous studies was costly and not scalable, or contained unnecessary tags or fusions.

Development of vaccines for preventing a future pandemic of severe acute respiratory syndrome (SARS) caused by SARS coronavirus (SARS-CoV) and for biodefense preparedness is urgently needed and encompassed herein. Previous studies have shown that a candidate SARS vaccine antigen that had the receptor-binding domain (RBD) of SARS-CoV spike protein can induce potent neutralizing antibody responses and protection against SARS-CoV challenge in vaccinated animals. To optimize expression conditions for scale-up production of the RBD vaccine candidate, it was considered that this is achievable by removing glycosylation sites in the RBD protein. In this disclosure, two RBD protein variants as examples were constructed: 1) RBDI93-WT (193-aa, residues 318-510) and its deglycosylated forms (RBDI93-N1, RBD193-N2, RBD193-N3); 2) RBD219-WT (219-aa, residues 318-536) and its deglycosylated forms (RBD219-N1, RBD219-N2, and RBD219-N3). Constructs can be expressed as recombinant proteins in yeast, as an example. The purified recombinant proteins of these constructs were compared for their antigenicity, functionality and immunogenicity in mice using alum as the adjuvant. RBD219-N1 exhibited higher expression yield, and maintained its antigenicity and functionality. More importantly, RBD219-N1 induced significantly stronger RBD-specific antibody responses and a higher level of neutralizing antibodies in immunized mice than RBDI93-WT, RBD193-N1, RBD193-N3 or RBD219-WT. Therefore, RBD219-N1 is useful as an optimal SARS immunogenic composition, such as a vaccine.

### II. SARS

Individuals provided with compositions and/or methods of the invention may be known to have SARS, may be suspected of having SARS, may be known to have been exposed to SARS, or may be suspected of having been exposed to SARS.

In the event an individual has contracted SARS, the first symptom is usually a fever of at least 38° C. (100.4° F.) or higher. Early symptoms last approximately 2-10 days and include generic flu-like symptoms, including chills/rigor, muscle aches, headaches, diarrhea, sore throat, runny nose, malaise, and muscle pain, for example. A dry cough, shortness of breath, and/or an upper respiratory tract infection may develop next. Lymphocyte counts in the blood are usually decreased, and platelet counts may also be low. Serum lactate dehydrogenase (LDH) and creatinine phosphokinase (CPK) levels may be increased. The individual may be subjected to a medical exam, chest x-ray and/or a HRCT scan as part of a diagnosis. In specific embodiments, diagnosis of SARS is an optional or required step in methods of the disclosure.

Severely affected people with SARS develop a potentially fatal form of respiratory failure, referred to as adult respiratory distress syndrome (ARD or ARDS). In such cases the virus attacks organs in the body other than the lungs, causing kidney failure, inflammation of the heart sac (pericarditis), severe systemic bleeding from disruption of clotting system (disseminated intravascular coagulation), reduced white blood cell counts (lymphopenia), inflammation of the arteries (vasculitis), or inflammation of the gut with diarrhea, for example.

In some methods of the invention, an individual is subjected to the step of identifying whether or not they have SARS. SARS-CoV may be detected using enzyme-linked immunoassays (ELISA) for its antibody or reverse transcriptase polymerase chain reaction (PCR) tests for its genetic materials, for example. Examples of tests includes those performed on respiratory secretions or blood.

An individual may be tested for SARS when they have the appropriate symptoms and/or who work with SARS-CoV in a laboratory or who have recent exposure to infected people or mammals.

### III. Middle East Respiratory Syndrome (MERS)

Individuals provided with compositions and/or methods of the invention may be known to have MERS, may be suspected of having MERS, may be known to have been exposed to MERS, or may be suspected of having been exposed to MERS.

MERS is viral respiratory illness first reported in Saudi Arabia in 2012. It is caused by a coronavirus called MERS-CoV (also termed EMC/2012 (HCoV-EMC/2012). Most people who have been confirmed to have MERS-CoV infection developed severe acute respiratory illness. They had fever, cough, and shortness of breath, and about half of these people died.

In some methods of the invention, an individual is subjected to the step of identifying whether or not they have MERS. Examples of tests includes those performed on respiratory secretions or blood, such as tests for a MERS antigen.

An individual may be tested for MERS when they have the appropriate symptoms and/or who work with MERS in a laboratory or who have recent exposure to infected people or mammals or when the individual is in or has been in a geographical location where individuals have MERS or are prone to have MERS. In specific embodiments, diagnosis of MERS is an optional or required step in methods of the disclosure.

### IV. Proteinaceous Vaccines and Immunogenic Compositions

In embodiments of the disclosure, a composition induces an immune response to the antigen in a cell, tissue or animal (e.g., a human). As used herein, an “antigenic composition” (which alternatively may be referred to as an “immunogenic composition”) may comprise an antigen (e.g., a protein, peptide, or polypeptide) or a modified version of an antigen. In particular embodiments the antigenic composition comprises or encodes all or part of the receptor-binding domain of the SARS CoV spike protein or a mutated version thereof, including a deglycosylated version thereof. In certain embodiments, the immunogenic composition or vaccine comprises at least one adjuvant. In other embodiments, the antigenic composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

SEQ ID NO:1 provides the nucleic acid sequence of SARS-CoV-RBD-193 (318-510aa), and SEQ ID NO:2 provides the amino acid sequence of it. SEQ ID NO:3 provides the nucleic acid sequence of SARS-CoV-RBD-219 (318-536aa), and SEQ ID NO:4 provides the amino acid sequence of it. An example of a full-length SARS-CoV spike protein is in GenBank® at DQ407820.1, which sequence is incorporated by reference herein in its entirety.

In certain embodiments, an antigenic composition or immunologically functional equivalent may be used as an effective vaccine in inducing an anti-SARS humoral and/or cell-mediated immune response in an animal, including human. The present invention contemplates one or more antigenic compositions or vaccines for use in both active and passive immunization embodiments.

A vaccine or immunogenic composition of the present invention may vary in its composition of proteinaceous components. It will be understood that various compositions described herein may further comprise additional components. For example, one or more vaccine or immunogenic composition components may be comprised in a lipid or liposome. In another non-limiting example, a vaccine or immunogenic composition may comprise one or more adjuvants. A vaccine or immunogenic composition of the present disclosure, and its various components, may be prepared and/or administered by any method disclosed herein or as would be known to one of ordinary skill in the art, in light of the present disclosure.

It is understood that an immunogenic composition may be made by a method that is well known in the art, including but not limited to chemical synthesis by solid phase synthesis and purification away from the other products of the chemical reactions by HPLC, or production by the expression of a nucleic acid sequence (e.g., a DNA sequence) encoding a peptide or polypeptide comprising an antigen of the present invention in an in vitro translation system or in a living cell including, for example, in a yeast cell, bacterial, mammalian cells or baculovirus/insect cells. The antigenic composition may be isolated and extensively purified to remove one or more undesired small molecular weight molecules and/or lyophilized for more ready formulation into a desired vehicle. It is further understood that amino acid additions, deletions, mutations, chemical modification and such like that are made in an antigenic composition component, such as a vaccine, will preferably not substantially interfere with the antibody recognition of the epitopic sequence.

A peptide or polypeptide corresponding to one or more antigenic determinants of the receptor binding domain of the SARS-CoV spike protein may generally be 10-20 amino acid residues in length, and may contain more than one peptide determinants or up to about 30-50 residues or so. A peptide sequence may be sythesized by methods known to those of ordinary skill in the art, such as, for example, peptide synthesis using automated peptide synthesis machines, such as those available from Applied Biosystems (Foster City, Calif.). In specific embodiments, the full-length peptide is an 193-aa fragment or a 219-aa fragment of SARS-CoV spike protein.

Longer peptides or polypeptides also may be prepared, e.g., by recombinant means. In certain embodiments, a nucleic acid encoding an antigenic composition and/or a component described herein may be used, for example, to produce an antigenic composition in vitro or in vivo for the various compositions and methods of the present invention. For example, in certain embodiments, a nucleic acid encoding an antigen is comprised in, for example, a vector in a recombinant cell. The nucleic acid may be expressed to produce a peptide or polypeptide comprising an antigenic sequence. The peptide or polypeptide may be secreted from the cell, or comprised as part of or within the cell.

A. Immunologically Functional Equivalents

As modifications and changes may be made in the structure of an antigenic composition of the present disclosure, and still obtain molecules having like or otherwise desirable characteristics, such immunologically functional equivalents are also encompassed within the present invention.

For example, certain amino acids may be substituted for other amino acids in a peptide, polypeptide or protein structure without appreciable loss of interactive binding capacity with structures such as, for example, antigen-binding regions of antibodies, binding sites on substrate molecules or receptors, DNA binding sites, or such like. Since it is the interactive capacity and nature of a peptide, polypeptide or protein that defines its biological (e.g., immunological) functional activity, certain amino acid sequence substitutions can be made in an amino acid sequence (or, of course, its underlying DNA coding sequence) and nevertheless obtain a peptide or polypeptide with like (agonistic) properties. It is thus contemplated by the inventors that various changes may be made in the sequence of an antigenic composition such as, for example a SARS Co-V RBD peptide or polypeptide without appreciable loss of biological utility or activity. In particular cases, one or more of the glycosylation sites of RBD is mutated or deleted and in particular embodiments there is also one or more other amino acids that are modified compared to the corresponding wild-type sequence.

As used herein, an “amino molecule” refers to any amino acid, amino acid derivative or amino acid mimic as would be known to one of ordinary skill in the art. In certain embodiments, the residues of the antigenic composition comprises amino molecules that are sequential, without any non-amino molecule interrupting the sequence of amino molecule residues. In other embodiments, the sequence may comprise one or more non-amino molecule moieties. In particular embodiments, the sequence of residues of the antigenic composition may be interrupted by one or more non-amino molecule moieties.

Accordingly, antigenic compositions, particularly an immunologically functional equivalent of the sequences disclosed herein, may encompass an amino molecule sequence comprising at least one of the 20 common amino acids in naturally synthesized proteins, or at least one modified or unusual amino acid.

In term of immunologically functional equivalent, it is well understood by the skilled artisan that, inherent in the definition is the concept that there is a limit to the number of changes that may be made within a defined portion of the molecule and still result in a molecule with an acceptable level of equivalent immunological activity. An immunologically functional equivalent peptide or polypeptide are thus defined herein as those peptide(s) or polypeptide(s) in which certain, not most or all, of the amino acid(s) may be substituted.

In particular, where a shorter length peptide is concerned, it is contemplated that fewer amino acid substitutions should be made within the given peptide. A longer polypeptide may have an intermediate number of changes. The full length protein will have the most tolerance for a larger number of changes. Of course, a plurality of distinct polypeptides/peptides with different substitutions may easily be made and used in accordance with the invention.

It also is well understood that where certain residues are shown to be particularly important to the immunological or structural properties of a protein or peptide, e.g., residues in binding regions or active sites, such residues may not generally be exchanged. This is an important consideration in the present invention, where changes in the antigenic site should be carefully considered and subsequently tested to ensure maintenance of immunological function (e.g., antigenicity), where maintenance of immunological function is desired. In this manner, functional equivalents are defined herein as those peptides or polypeptides which maintain a substantial amount of their native immunological activity.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as immunologically functional equivalents.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, polypeptide or peptide is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity, particularly where the immunological functional equivalent polypeptide or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a immunological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

Numerous scientific publications have also been devoted to the prediction of secondary structure, and to the identification of an epitope, from analyses of an amino acid sequence (Chou & Fasman, 1974a,b; 1978a,b, 1979). Any of these may be used, if desired, to supplement the teachings of U.S. Pat. No. 4,554,101.

Moreover, computer programs are currently available to assist with predicting an antigenic portion and an epitopic core region of one or more proteins, polypeptides or peptides. Examples include those programs based upon the Jameson-Wolf analysis (Jameson & Wolf, 1988; Wolf et al., 1988), the program PepPlot® (Brutlag et al., 1990; Weinberger et al., 1985), and other new programs for protein tertiary structure prediction (Fetrow & Bryant, 1993). Another commercially available software program capable of carrying out such analyses is MacVector (IBI, New Haven, Conn.).

In further embodiments, major antigenic determinants of a peptide or polypeptide may be identified by an empirical approach in which portions of a nucleic acid encoding a peptide or polypeptide are expressed in a recombinant host, and the resulting peptide(s) or polypeptide(s) tested for their ability to elicit an immune response. For example, PCR™ can be used to prepare a range of peptides or polypeptides lacking successively longer fragments of the C-terminus of the amino acid sequence. The immunoactivity of each of these peptides or polypeptides is determined to identify those fragments or domains that are immunodominant. Further studies in which only a small number of amino acids are removed at each iteration then allows the location of the antigenic determinant(s) of the peptide or polypeptide to be more precisely determined.

Another method for determining a major antigenic determinant of a peptide or polypeptide is the SPOTs™ system (Genosys Biotechnologies, Inc., The Woodlands, Tex.). In this method, overlapping peptides are synthesized on a cellulose membrane, which following synthesis and deprotection, is screened using a polyclonal or monoclonal antibody. An antigenic determinant of the peptides or polypeptides which are initially identified can be further localized by performing subsequent syntheses of smaller peptides with larger overlaps, and by eventually replacing individual amino acids at each position along the immunoreactive sequence.

Once one or more such analyses are completed, an antigenic composition, such as for example a peptide or a polypeptide is prepared that contain at least the essential features of one or more antigenic determinants. An antigenic composition is then employed in the generation of antisera against the composition, and preferably the antigenic determinant(s).

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid. Nucleic acids encoding these antigenic compositions also can be constructed and inserted into one or more expression vectors by standard methods (Sambrook et al., 1987), for example, using PCR™ cloning methodology.

In addition to the peptidyl compounds described herein, the inventors also contemplate that other sterically similar compounds may be formulated to mimic the key portions of the peptide or polypeptide structure or to interact specifically with, for example, an antibody. Such compounds, which may be termed peptidomimetics, may be used in the same manner as a peptide or polypeptide of the invention and hence are also immunologically functional equivalents.

Certain mimetics that mimic elements of protein secondary structure are described in Johnson et al. (1993). The underlying rationale behind the use of peptide mimetics is that the peptide backbone of proteins exists chiefly to orientate amino acid side chains in such a way as to facilitate molecular interactions, such as those of antibody and antigen. A peptide mimetic is thus designed to permit molecular interactions similar to the natural molecule.

B. Antigen Mutagenesis

In particular embodiments, an antigenic composition is mutated for purposes such as, for example, enhancing its immunogenicity or producing or identifying a immunologically functional equivalent sequence. Methods of mutagenesis are well known to those of skill in the art (Sambrook et al., 1987).

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In a preferred embodiment, site directed mutagenesis is used. Site-specific mutagenesis is a technique useful in the preparation of an antigenic composition, through specific mutagenesis of the underlying DNA. In general, the technique of site-specific mutagenesis is well known in the art. The technique further provides a ready ability to prepare and test sequence variants, incorporating one or more of the foregoing considerations, by introducing one or more nucleotide sequence changes into the DNA. Site-specific mutagenesis allows the production of a mutant through the use of specific oligonucleotide sequence(s) which encode the DNA sequence of the desired mutation, as well as a sufficient number of adjacent nucleotides, to provide a primer sequence of sufficient size and sequence complexity to form a stable duplex on both sides of the position being mutated. Typically, a primer of about 17 to about 75 nucleotides in length is preferred, with about 10 to about 25 or more residues on both sides of the position being altered, while primers of about 17 to about 25 nucleotides in length being more preferred, with about 5 to 10 residues on both sides of the position being altered.

In general, site-directed mutagenesis is performed by first obtaining a single-stranded vector, or melting of two strands of a double stranded vector which includes within its sequence a DNA sequence encoding the desired protein. As will be appreciated by one of ordinary skill in the art, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

This mutagenic primer is then annealed with the single-stranded DNA preparation, and subjected to DNA polymerizing enzymes such as, for example, E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

Alternatively, a pair of primers may be annealed to two separate strands of a double stranded vector to simultaneously synthesize both corresponding complementary strands with the desired mutation(s) in a PCR™ reaction. A genetic selection scheme to enrich for clones incorporating the mutagenic oligonucleotide has been devised (Kunkel et al., 1987). Alternatively, the use of PCR™ with commercially available thermostable enzymes such as Taq polymerase may be used to incorporate a mutagenic oligonucleotide primer into an amplified DNA fragment that can then be cloned into an appropriate cloning or expression vector (Tomic et al., 1990; Upender et al., 1995). A PCR™ employing a thermostable ligase in addition to a thermostable polymerase also may be used to incorporate a phosphorylated mutagenic oligonucleotide into an amplified DNA fragment that may then be cloned into an appropriate cloning or expression vector (Michael 1994).

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

Additionally, one particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989).

C. Liposome-Mediated Transfection

In a further embodiment of the invention, one or more vaccine or immunogenic composition components may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991).

D. Vaccine or Immunogenic Composition Component Purification

In any case, a vaccine component (e.g., an antigenic peptide or polypeptide) may be isolated and/or purified from the chemical synthesis reagents, cell or cellular components. In a method of producing the vaccine or immunogenic composition component, purification is accomplished by any appropriate technique that is described herein or well-known to those of skill in the art (e.g., Sambrook et al., 1987). There is no general requirement that an antigenic composition of the present invention or other vaccine component always be provided in their most purified state. Indeed, it is contemplated that less substantially purified vaccine or immunogenic composition component, which is nonetheless enriched in the desired compound, relative to the natural state, will have utility in certain embodiments, such as, for example, total recovery of protein product, or in maintaining the activity of an expressed protein. However, it is contemplated that inactive products also have utility in certain embodiments, such as, e.g., in determining antigenicity via antibody generation.

The present invention also provides purified, and in certain embodiments, substantially purified vaccines or immunogenic composition components. The term “purified vaccine component” or “purified immunogenic composition component” as used herein, is intended to refer to at least one respective vaccine or immunogenic composition component (e.g., a proteinaceous composition, isolatable from cells), wherein the component is purified to any degree relative to its naturally-obtainable state, e.g., relative to its purity within a cellular extract or reagents of chemical synthesis. In certain aspects wherein the vaccine component is a proteinaceous composition, a purified vaccine component also refers to a wild-type or mutant protein, polypeptide, or peptide free from the environment in which it naturally occurs.

Where the term “substantially purified” is used, this will refer to a composition in which the specific compound (e.g., a protein, polypeptide, or peptide) forms the major component of the composition, such as constituting about 50% of the compounds in the composition or more. In preferred embodiments, a substantially purified vaccine component will constitute more than about 60%, about 70%, about 80%, about 90%, about 95%, about 99% or even more of the compounds in the composition.

In certain embodiments, a vaccine or immunogenic composition component may be purified to homogeneity. As applied to the present invention, “purified to homogeneity,” means that the vaccine component has a level of purity where the compound is substantially free from other chemicals, biomolecules or cells. For example, a purified peptide, polypeptide or protein will often be sufficiently free of other protein components so that degradative sequencing may be performed successfully. Various methods for quantifying the degree of purification of a vaccine component will be known to those of skill in the art in light of the present disclosure. These include, for example, determining the specific protein activity of a fraction (e.g., antigenicity), or assessing the number of polypeptides within a fraction by gel electrophoresis.

Various techniques suitable for use in chemical, biomolecule or biological purification, well known to those of skill in the art, may be applicable to preparation of a vaccine component of the present invention. These include, for example, precipitation with ammonium sulfate, PEG, antibodies and the like or by heat denaturation, followed by centrifugation; fractionation, chromatographic procedures, including but not limited to, partition chromatograph (e.g., paper chromatograph, thin-layer chromatograph (TLC), gas-liquid chromatography and gel chromatography) gas chromatography, high performance liquid chromatography, affinity chromatography, supercritical flow chromatography ion exchange, gel filtration, reverse phase, hydroxylapatite, lectin affinity; isoelectric focusing and gel electrophoresis (see for example, Sambrook et al. 1989; and Freifelder, Physical Biochemistry, Second Edition, pages 238-246, incorporated herein by reference).

Given many DNA and proteins are known (see for example, the National Center for Biotechnology Information's GenBank® and GenPept® databases, or may be identified and amplified using the methods described herein, any purification method for recombinately expressed nucleic acid or proteinaceous sequences known to those of skill in the art can now be employed. In certain aspects, a nucleic acid may be purified on polyacrylamide gels, and/or cesium chloride centrifugation gradients, or by any other means known to one of ordinary skill in the art (see for example, Sambrook et al. 1989, incorporated herein by reference). In further aspects, a purification of a proteinaceous sequence may be conducted by recombinately expressing the sequence as a fusion protein. Such purification methods are routine in the art. This is exemplified by the generation of an specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. In particular aspects, cells or other components of the vaccine may be purified by flow cytometry. Flow cytometry involves the separation of cells or other particles in a liquid sample, and is well known in the art (see, for example, U.S. Pat. Nos. 3,826,364, 4,284,412, 4,989,977, 4,498,766, 5,478,722, 4,857,451, 4,774,189, 4,767,206, 4,714,682, 5,160,974 and 4,661,913). Any of these techniques described herein, and combinations of these and any other techniques known to skilled artisans, may be used to purify and/or assay the purity of the various chemicals, proteinaceous compounds, nucleic acids, cellular materials and/or cells that may comprise a vaccine of the present invention. As is generally known in the art, it is believed that the order of conducting the various purification steps may be changed, or that certain steps may be omitted, and still result in a suitable method for the preparation of a substantially purified antigen or other vaccine component.

E. Additional Vaccine Components

It is contemplated that an antigenic composition of the invention may be combined with one or more additional components to form a more effective composition or vaccine. Non-limiting examples of additional components include, for example, one or more additional antigens, immunomodulators or adjuvants to stimulate an immune response to an antigenic composition of the present invention and/or the additional component(s).

1. Immunomodulators

For example, it is contemplated that immunomodulators can be included in the vaccine to augment a cell's or a patient's (e.g., an animal's) response. Immunomodulators can be included as purified proteins, nucleic acids encoding immunomodulators, and/or cells that express immunomodulators in the vaccine composition, for example. The following sections list non-limiting examples of immunomodulators that are of interest, and it is contemplated that various combinations of immunomodulators may be used in certain embodiments (e.g., a cytokine and a chemokine).

Interleukins, cytokines, nucleic acids encoding interleukins or cytokines, and/or cells expressing such compounds are contemplated as possible vaccine components. Interleukins and cytokines, include but are not limited to interleukin 1 (IL-1), IL-2, IL-3, IL-4, IL-5, IL-6, IL-7, IL-8, IL-9, IL-10, IL-11, IL-12, IL-13, IL-14, IL-15, IL-18, β-interferon, α-interferon, γ-interferon, angiostatin, thrombospondin, endostatin, GM-CSF, G-CSF, M-CSF, METH-1, METH-2, tumor necrosis factor, TGFβ, LT and combinations thereof.

Chemokines, nucleic acids that encode for chemokines, and/or cells that express such also may be used as vaccine components. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-alpha, MIP1-Beta, IP-10 and combinations thereof. The skilled artisan will recognize that certain cytokines are also known to have chemoattractant effects and could also be classified under the term chemokines.

In certain embodiments, an antigenic composition may be chemically coupled to a carrier or recombinantly expressed with a immunogenic carrier peptide or polypetide (e.g., a antigen-carrier fusion peptide or polypeptide) to enhance an immune reaction. Exemplary and preferred immunogenic carrier amino acid sequences include hepatitis B surface antigen, keyhole limpet hemocyanin (KLH) and bovine serum albumin (BSA). Other albumins such as ovalbumin, mouse serum albumin or rabbit serum albumin also can be used as immunogenic carrier proteins. Means for conjugating a polypeptide or peptide to a immunogenic carrier protein are well known in the art and include, for example, glutaraldehyde, m-maleimidobenzoyl-N-hydroxysuccinimide ester, carbodiimide and bis-biazotized benzidine.

It may be desirable to coadminister biologic response modifiers (BRM), which have been shown to upregulate T cell immunity or downregulate suppressor cell activity. Such BRMs include, but are not limited to, cimetidine (CIM; 1200 mg/d) (Smith/Kline, PA); low-dose cyclophosphamide (CYP; 300 mg/m²) (Johnson/Mead, NJ), or a gene encoding a protein involved in one or more immune helper functions, such as B-7.

2. Adjuvants

Immunization protocols have used adjuvants to stimulate responses for many years, and as such adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation.

In one aspect, an adjuvant effect is achieved by use of an agent, such as alum, used in about 0.05 to about 0.1% solution in phosphate buffered saline. Alternatively, the antigen is made as an admixture with synthetic polymers of sugars (Carbopol®) used as an about 0.25% solution. Adjuvant effect may also be made my aggregation of the antigen in the vaccine by heat treatment with temperatures ranging between about 70° to about 101° C. for a 30-second to 2-minute period, respectively. Aggregation by reactivating with pepsin treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA®) used as a block substitute, also may be employed.

Some adjuvants, for example, certain organic molecules obtained from bacteria, act on the host rather than on the antigen. An example is muramyl dipeptide (N-acetylmuramyl-L-alanyl-D-isoglutamine [MDP]), a bacterial peptidoglycan. The effects of MDP, as with most adjuvants, are not fully understood. MDP stimulates macrophages but also appears to stimulate B cells directly. The effects of adjuvants, therefore, are not antigen-specific. If they are administered together with a purified antigen, however, they can be used to selectively promote the response to the antigen.

Adjuvants have been used experimentally to promote a generalized increase in immunity against unknown antigens (e.g., U.S. Pat. No. 4,877,611). In certain embodiments, hemocyanins and hemoerythrins may also be used in the invention. The use of hemocyanin from keyhole limpet (KLH) is preferred in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described (Yin et al., 1989). The doses that produce optimal responses, or that otherwise do not produce suppression, should be employed as indicated (Yin et al., 1989). Polyamine varieties of polysaccharides are particularly preferred, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide which is described for use in artificial liposomes formed from phosphatidyl choline and phosphatidyl glycerol. It is the to be effective in activating human monocytes and destroying tumor cells, but is non-toxic in generally high doses. The compounds of U.S. Pat. No. 4,950,645 and PCT Patent Application WO 91/16347, are contemplated for use with cellular carriers and other embodiments of the present invention.

Another adjuvant contemplated for use in the present invention is BCG. BCG (bacillus Calmette-Guerin, an attenuated strain of Mycobacterium) and BCG-cell wall skeleton (CWS) may also be used as adjuvants in the invention, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945.

BCG is an important clinical tool because of its immunostimulatory properties. BCG acts to stimulate the reticulo-endothelial system, activates natural killer cells and increases proliferation of hematopoietic stem cells. Cell wall extracts of BCG have proven to have excellent immune adjuvant activity. Molecular genetic tools and methods for mycobacteria have provided the means to introduce foreign genes into BCG (Jacobs et al., 1987; Snapper et al., 1988; Husson et al., 1990; Martin et al., 1990).

Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man (Luelmo, 1982; Lotte et al., 1984). It is one of the few vaccines that can be given at birth, it engenders long-lived immune responses with only a single dose, and there is a worldwide distribution network with experience in BCG vaccination. An exemplary BCG vaccine is sold as TICE® BCG (Organon Inc., West Orange, N.J.).

Amphipathic and surface active agents, e.g., saponin and derivatives such as QS21 (Cambridge Biotech), form yet another group of adjuvants for use with the immunogens of the present invention. Nonionic block copolymer surfactants (Rabinovich et al., 1994; Hunter et al., 1991) may also be employed. Oligonucleotides are another useful group of adjuvants (Yamamoto et al., 1988). Quil A and lentinen are other adjuvants that may be used in certain embodiments of the present invention.

One group of adjuvants preferred for use in the invention are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins, is also envisioned to be useful, as described in U.S. Pat. No. 4,520,019.

In other embodiments, the present invention contemplates that a variety of adjuvants may be employed in the membranes of cells, resulting in an improved immunogenic composition. The only requirement is, generally, that the adjuvant be capable of incorporation into, physical association with, or conjugation to, the cell membrane of the cell in question. Those of skill in the art will know the different kinds of adjuvants that can be conjugated to cellular vaccines in accordance with this invention and these include alkyl lysophosphilipids (ALP); BCG; and biotin (including biotinylated derivatives) among others. Certain adjuvants particularly contemplated for use are the teichoic acids from Gram-cells. These include the lipoteichoic acids (LTA), ribitol teichoic acids (RTA) and glycerol teichoic acid (GTA). Active forms of their synthetic counterparts may also be employed in connection with the invention (Takada et al., 1995a).

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

One group of adjuvants preferred for use in some embodiments of the present invention are those that can be encoded by a nucleic acid (e.g., DNA or RNA). It is contemplated that such adjuvants may be encoded in a nucleic acid (e.g., an expression vector) encoding the antigen, or in a separate vector or other construct. These nucleic acids encoding the adjuvants can be delivered directly, such as for example with lipids or liposomes.

3. Excipients, Salts and Auxiliary Substances

An antigenic composition of the present invention may be mixed with one or more additional components (e.g., excipients, salts, etc.) which are pharmaceutically acceptable and compatible with at least one active ingredient (e.g., antigen). Suitable excipients are, for example, water, saline, dextrose, glycerol, ethanol and combinations thereof.

An antigenic composition of the present invention may be formulated into the vaccine as a neutral or salt form. A pharmaceutically-acceptable salt, includes the acid addition salts (formed with the free amino groups of the peptide) and those which are formed with inorganic acids such as, for example, hydrochloric or phosphoric acid, or such organic acids as acetic, oxalic, tartaric, mandelic, and the like. A salt formed with a free carboxyl group also may be derived from an inorganic base such as, for example, sodium, potassium, ammonium, calcium, or ferric hydroxide, and such organic bases as isopropylamine, trimethylamine, 2-ethylamino ethanol, histidine, procaine, and combinations thereof.

In addition, if desired, an antigentic composition may comprise minor amounts of one or more auxiliary substances such as for example wetting or emulsifying agents, pH buffering agents, etc. which enhance the effectiveness of the antigenic composition or vaccine.

F. Vaccine and Immunogenic Composition Preparations

Once produced, synthesized and/or purified, an antigen or other vaccine component may be prepared as a vaccine or immunogenic composition for administration to an individual. The preparation of a vaccine is generally well understood in the art, as exemplified by U.S. Pat. Nos. 4,608,251, 4,601,903, 4,599,231, 4,599,230, and 4,596,792, all incorporated herein by reference. Such methods may be used to prepare a vaccine comprising an antigenic composition comprising a particular RBD of SARS-CoV as active ingredient(s), in light of the present disclosure. In particular embodiments, the compositions of the present invention are prepared to be pharmacologically acceptable vaccines.

Pharmaceutical vaccine or immunogenic compositions of the present invention comprise an effective amount of one or more certain RBDs of SARS-CoV dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one RBD of SARS-CoV will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). The modified RBD of SARS-CoV may comprise different types of carriers depending on whether it is to be administered in solid, liquid or aerosol form, and whether it need to be sterile for such routes of administration as injection. Except insofar as any conventional carrier is incompatible with the active ingredient, its use in the therapeutic or pharmaceutical compositions is contemplated.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The modified RBD of SARS-CoV may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present disclosure. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the RBD of SARS-CoV is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

G. Vaccine or Immunogenic Composition Administration

The manner of administration of a vaccine or immunogenic composition may be varied widely. Any of the conventional methods for administration of a vaccine or immunogenic composition are applicable. For example, a vaccine may be conventionally administered intravenously, intradermally, intraarterially, intraperitoneally, intralesionally, intracranially, intraarticularly, intraprostaticaly, intrapleurally, intratracheally, intranasally, intravitreally, intravaginally, intratumorally, intramuscularly, intraperitoneally, subcutaneously, intravesicularlly, mucosally, intrapericardially, orally, rectally, nasally, topically, in eye drops, locally, using aerosol, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

A vaccination or immunogenic composition delivery schedule and dosages may be varied on a patient by patient basis, taking into account, for example, factors such as the weight and age of the patient, the type of disease being treated, the severity of the disease condition, previous or concurrent therapeutic interventions, the manner of administration and the like, which can be readily determined by one of ordinary skill in the art.

A vaccine or immunogenic composition may be administered in a manner compatible with the dosage formulation, and in such amount as will be therapeutically effective and immunogenic. For example, the intramuscular route may be preferred in the case of toxins with short half lives in vivo. The quantity to be administered depends on the subject to be treated, including, e.g., the capacity of the individual's immune system to synthesize antibodies, and the degree of protection desired. The dosage of the vaccine will depend on the route of administration and will vary according to the size of the host. Precise amounts of an active ingredient required to be administered depend on the judgment of the practitioner. In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein However, a suitable dosage range may be, for example, of the order of several hundred micrograms active ingredient per vaccination. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per vaccination, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. A suitable regime for initial administration and booster administrations (e.g., innoculations) are also variable, but are typified by an initial administration followed by subsequent inoculation(s) or other administration(s).

In many instances, it will be desirable to have multiple administrations of the vaccine or immunogenic composition, usually not exceeding six vaccinations, for example, more usually not exceeding four vaccinations and in some cases one or more, usually at least about three vaccinations. The vaccinations may be at from two to twelve week intervals, more usually from three to five week intervals, although longer intervals are encompassed herein. Periodic boosters at intervals of 1-5 years, usually three years, may be desirable to maintain protective levels of the antibodies.

The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays. Other immune assays can be performed and assays of protection from challenge with the RBD of SARS-CoV can be performed, following immunization.

### V. Kits of the Disclosure

Any of the compositions described herein may be comprised in a kit. In a non-limiting example, a modified RBD SARS-CoV spike composition may be comprised in a kit. In a non-limiting example, an immunogenic composition comprising a modified RBD SARS-CoV spike composition may be comprised in a kit. In a non-limiting example, a vaccine comprising a modified RBD SARS-CoV spike composition may be comprised in a kit, including deglycosylated.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

The component(s) of the kit may be provided as dried powder(s). When reagents and/or components are provided as a dry powder, the powder can be reconstituted by the addition of a suitable solvent. It is envisioned that the solvent may also be provided in another container means. The kits may comprise a container means for containing a sterile, pharmaceutically acceptable buffer and/or other diluent.

Irrespective of the number and/or type of containers, the kits of the invention may also comprise, and/or be packaged with, an instrument for assisting with the injection/administration and/or placement of the ultimate composition within the body of an animal. Such an instrument may be a syringe, pipette, forceps, and/or any such medically approved delivery vehicle. In some cases, there are one or more means to identify the presence of SARS in a sample from an individual.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials and Methods

Cloning and Expression of RBDs in Yeast Pichia pastoris

The DNAs encoding the 193-aa (RBD193, residues 318-510) and 219-aa (RBD219, residues 318-536) of SARS-CoV RBD were codon-optimized based on yeast codon usage preference and synthesized by GenScript (Piscataway, N.J.), followed by subcloning into Pichia secretory expression vector pPICZαA (Invitrogen, Grand Island, N.Y.) using EcoRI/XbaI restriction sites. The correct insert sequences and reading frames of recombinant plasmids were confirmed by double-stranded sequencing using vector flanking primers α-factor and 3′AOX-1. The recombinant plasmid DNAs were then transformed into Pichia pastoris X-33 by electroporation. The expressions of recombinant RBD193 and RBD219 were induced with 0.5% methanol at 30° C. for 72 h, and the highest expression clone was chosen to make seed stock in 20% glycerol as described previously (He et al., 2005).

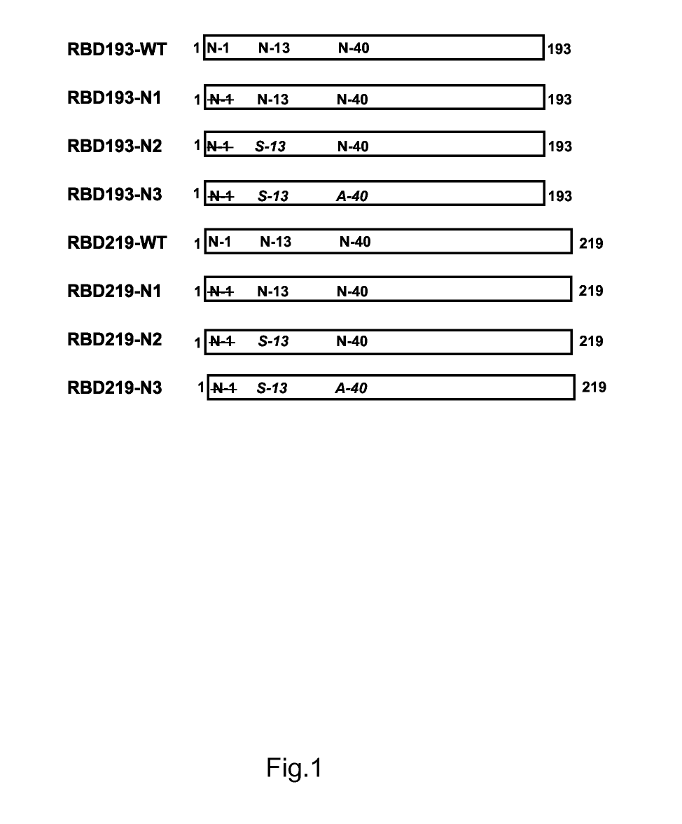

Because high glycosylation of the RBD193/RBD219-wild-type (WT) expressed in yeast could cause yield and reproducibility problems, the three asparagines in the RBD sequence responsible for N-glycosylation were removed or mutated to make deglycosylated forms as follows: N1: deletion of the 1st Asn (N-1), the 1st N-glycosylation site; N2: mutation of the 2nd N-glycosylated Asn (N-13) to Ser in addition to the Ni deletion; and N3: mutation of the 3rd N-glycosylated N-40 to Ala, in addition of N1 and N2 deletion/mutation (FIG. 1). The expression and glycosylation level of the recombinant RBD193-WT and RBD219-WT, and their deglycosylated forms was confirmed by SDS-PAGE and Western blot with anti-RBD monoclonal antibody (mAb) 33G4 developed in the laboratory (Goud et al., 2004).

Glycosidase Assay

In order to determine whether the expressed recombinant RBD193-WT is glycosylated, yeast-expressed RBD193-WT was digested with Peptide-N-Glycosidase F (PNGase F) (New England Biolabs (NEB), Ipswich, Mass.). Briefly, 10 μl of the RBD193-WT/pPICZαA/P. pastoris culture induced with 0.5% methanol for 72 h were mixed with 1 μl of denaturing buffer (NEB) in a 1.5 ml tube and denatured at 100° C. for 10 min. Then 2 μl of G7 buffer, 2 μl of 10% NP40 (all came with PNGase F), 1 μl of N-PNGase F, and 5 μl of deionized water were added in the tube. The mixture was then incubated at 37° C. for an hour. The removal of glycans was confirmed by SDS-PAGE, followed by Western blot using anti-RBD mAb 33G4.

Optimization of Induction Conditions by pH and Detergent

Seed of RBD193-WT/pPICZαA/P. pastoris X-33 was grown in 5 ml of Buffered Glycerol-complex Medium (BMGY) at 225 rpm at 30° C. overnight until the OD600 reached 2-6. The expression of recombinant RBD193-WT was induced in 10 ml Buffered Methanol-complex Medium (BMMY) (starting OD600=1.0) containing 0.5% methanol with different pH of 5.2, 6.0, 7.5, and 8.0. EMPIGEN® BB Detergent was added into the culture (pH 6.0) at final concentration of 0.01% and 0.05% in order to determine if the detergent was able to disrupt any possible aggregation of the expressed recombinant protein. The induction was continued for 72 h. The expression, yield, and integrity of the expressed recombinant RBD193 in different culture media were identified by Western blot using anti-RBD mAb 33G4.

Fermentation and Purification

To scale-up the expression of recombinant RBDs in yeast, the constructs of RBD193-N1, RBD193-N3, RBD219-WT, and RBD219-N1 in pPICZαA/P. pastoris X33 were fermented in 5 L fermentation as described previously (Du et al., 2009). Briefly, the seed stocks of each construct were used to inoculate 1 L of Buffered Minimal Glycerol (BMG) medium and cultured overnight at 37° C. with shaking at 225 rpm until the OD600 reached ˜10.0. 110 ml of this culture were used to inoculate 2.5 L of sterile BSM in the fermenter containing 3.5 ml/L of PTM1 trace elements and 3.5 ml/L of 0.02% d-Biotin. Fermentation was initiated at 30° C., and the initial pH was set at 5.0. Gas and agitation were adjusted to maintain dissolved oxygen (DO) at 30%. Upon exhaustion of glycerol during the batch phase (DO spike), methanol was pumped in from 0.8 ml/L/h to 10 ml/Uh over a 6-8 hour period, the pH was adjusted to 6.0 using 14% ammonium hydroxide, and the induction was maintained at 26° C. for 75 h. After fermentation, the culture was harvested by centrifugation at 7,000 rpm for 30 minutes at 4° C. and filtered through a 0.22 pm bottle top filtration unit. The expression yield of recombinant RBDs in the fermented culture was determined by SDS-PAGE and densitometry. To purify RBD193N1, RBD219-WT, and RBD219-N1, one part of fermentation supernatant was diluted with 2 parts of 30 mM Tris and 3 M ammonium sulfate, at pH 8.0, and then loaded to HiTrap Butyl Sepharose HP at the flow rate of 1.5 ml/min, followed by washing with 30 mM Tris and 2 M ammonium sulfate to remove the unbound proteins. The bound RBD protein was eluted with gradient ammonium sulfate starting from 2 M. The fractions containing target protein were pooled together, concentrated, and further purified with Toyopearl HW55S size exclusion column in order to eliminate the remaining contaminants. To purify RBD193-N3, the fermented culture supernatant was chromatographed using an anion exchange Q Sepharose XL column. The flow-through was collected, concentrated and, like other RBD proteins, further purified with Toyopearl HW55S size exclusion column. The purity of recombinant RBDs was confirmed by SDS-PAGE and Western Blot using anti-RBD mAbs 33G4, 35B5, 24H8, and 31H12 developed in the laboratory (Goud et al., 2004).

Animals

Four- to six-week-old female BALB/c mice were used for the study and housed in the animal facility of the New York Blood Center. The animal studies were carried out in accordance with the recommendations for the Care and Use of Laboratory Animals of the National Institutes of Health. The animal protocol was approved by the Committee on the Ethics of Animal Experiments of the New York Blood Center (Permit Number: 194.14).

Mouse Vaccination and Sera Collection

The immunization protocols were performed as previously described with some modifications (Du et al., 2009; Du et al., 2010; Du et al., 2009). Briefly, mice were subcutaneously (s.c.) immunized with yeast-expressed recombinant RBD proteins (RBD193-N1, RBD193-N3, RBD219-N1, or RBD219-WT) (20 μg/mouse) formulated with Alhydrogel 2% (aluminium hydroxide gel, hereinafter alum) adjuvant (InvivoGen, San Diego, Calif.). A mammalian cell 293T-expressing SARS-CoV RBD protein (RBD193-WT) (Du et al., 2009) and PBS were used as positive and negative control, respectively. Immunized mice were boosted twice with the same alum-formulated immunogen (10 μg/mouse) at 21-day intervals. Mouse sera were collected before immunization and 10 days after each vaccination to measure humoral IgG antibody responses and neutralizing antibodies.

ELISA

ELISA was used to verify the conformation of the yeast-expressed SARS-CoV RBD proteins to RBD-specific mAbs developed in the laboratory (Goud et al., 2004). Briefly, 96-well ELISA plates were respectively pre-coated with each yeast-expressed RBD protein (1 μg/ml) overnight at 4° C. and blocked with 2% non-fat milk for 2 h at 37° C. A series of conformation-dependent mAbs, including 24H8 (Conf I), 31H12 (Conf II), 35B5 (Conf IV), 33G4 (Conf V), 19B2 (Conf VI), and linear-dependent mAb 17H9 (Goud et al., 2004), were added to the plates and incubated for 1 h at 37° C., followed by four washes. Bound antibodies were reacted with horseradish peroxidase (HRP)-conjugated anti-mouse IgG (1:3,000, Invitrogen) for 1 h at 37° C. After four washes, the substrate 3,3′,5,5′-tetramethylbenzidine (TMB) (Zymed) was added to the plates, and the reaction was stopped by adding 1 N H2SO4. The absorbance at 450 nm (A450) was measured with an ELISA plate reader (Tecan, San Jose, Calif.).

In addition, the reactivity of polyclonal antibodies in the collected mouse sera against the expressed RBD was also measured by ELISA using previously described protocols with some modifications (Du et al., 2009; Du et al., 2010; Du et al., 2009). Briefly, 96-well ELISA plates were respectively pre-coated with yeast-expressed RBD219-WT protein (1 μg/ml) overnight at 4° C. and then serially diluted mouse sera was added. Bound IgG antibody was detected by using HRP-conjugated anti-mouse IgG (1:2,000), followed by the same protocol as described above.

Pull-Down Binding Assay

The binding of yeast-expressed recombinant SARS-CoV RBD proteins to cell-associated receptor ACE2 or soluble ACE2 (sACE2) (R&D Systems, Minneapolis, Minn.) was performed by pull-down assay using previously described protocols with some modifications (Du et al., 2013; Du et al., 2013). Briefly, the lysates of 293T cells expressing ACE2 (ACE2/293T) or sACE2 were respectively incubated with recombinant SARS-CoV RBD proteins plus 17H9 linear mAb (Goud et al., 2004) and Protein A and G (for cell-associated ACE2) or Ni-NTA affinity column (for sACE2). After rotation at 4° C. overnight, the supernatant of the mixture was removed by centrifugation. After washing with PBS 3 times, the pellet with binding proteins was boiled for 10 min and the supernatants subjected to SDS-PAGE and Western blot as described below.

SDS-PAGE and Western Blot

SDS-PAGE and Western blot were performed using previously described protocols with some modifications (Du et al., 2011; Du et al., 2008; Du et al., 2008). Briefly, the pull-down proteins were subjected to SDS-PAGE and transferred to a nitrocellulose membrane, which was blocked with 5% non-fat milk in PBS with 0.05% Tween-20 (PBST) at 4° C. overnight, followed by successive incubation with goat anti-ACE2 mAb (1 μg/ml) and HRP-labeled anti-goat IgG (1:1,000, R&D Systems) for 1 h at room temperature. Signals were visualized with ECL Western blot substrate reagents (GE Healthcare, Piscataway, N.J.) and Amersham Hyperfilm (GE Healthcare).

Pseudovirus Neutralization Assay

Neutralizing antibody titers of recombinant RBD-immunized mouse sera were measured using a pseudovirus neutralization assay as previously described with some modifications (Du et al., 2009; Du et al., 2009). Briefly, 293T cells were co-transfected with a plasmid encoding SARS-CoV S protein and a plasmid encoding Env-defective, luciferase-expressing HIV-1 genome (pNL4-3.luc.RE), using the calcium phosphate method. The culture supernatant was harvested 72 h post-transfection and used for single-cycle infection of 293T cells expressing SARS-CoV's receptor ACE2 (ACE2/293T). The cells were seeded in 96-well culture plates at 104/well and incubated at 37° C. for 4-6 h to forma monolayer. Serial 2-fold diluted mouse sera were mixed with SARS-CoV pseudovirus at 37° C. for 1 h and then transferred to the monolayer cells. After incubation for 72 h, relative luciferase activity was measured by Ultra 384 luminometer (Tecan). SARS pseudovirus neutralization was calculated and expressed as 50% neutralizing antibody titer (NT50).

Live Virus-Based Neutralization Assay

The neutralizing titers of recombinant RBD-immunized mouse sera were further measured using a live virus-based neutralization assay as previously described with some modifications (He et al., 2004; He et al., 2005). Briefly, serial 2-fold diluted mouse sera were mixed with ˜100 infectiousSARS-CoV at 37° C. for 1 h and then added to the monolayer of Vero E6 cells in duplicate. Cytopathic effect (CPE) in each well was observed daily and recorded on day3 post-infection. The neutralizing titers were reported as the reciprocal of the highest dilution of serum that completely prevented CPE in at least 50% of the wells (NT50).

### Example 2

### Expression of Recombinant RBDS in P. Pastoris

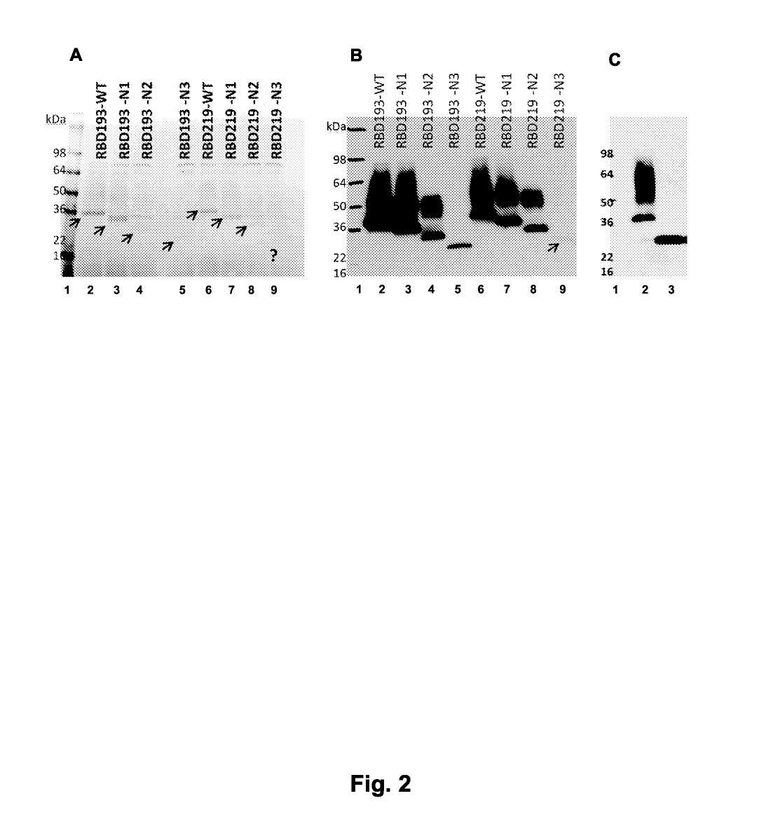

Different constructs of RBD193 and RBD219 (WT, N1, N2 and N3) were transformed into P. pastoris X-33, and 20 clones from each transformation were induced for recombinant protein expression in 10 ml tubes with 0.5% methanol. After induction for 72 h, recombinant RBDs with different size for different constructs were observed by SDS-PAGE Coomassie-stained gels and Western blot with anti-RBD mAb 33G4. The apparent molecular weight (M.W.) of recombinant RBDs was higher than expected based on the sequence, and a high M.W. smear was observed, especially in wild-type (WT) constructs, in both RBDI93 and RBD219, indicating that the recombinant RBD-WTs were glycosylated or aggregated (FIGS. 2A and 2B). The extent of glycosylation of RBD193-WT was confirmed by digesting the protein with N-glycosidase PNGase F. After digestion, the high M.W. smear disappeared, and the size of RBD193-WT returned to the expected M.W. (23 kDa) (FIG. 2C). This assay also confirmed that the high M.W. smear was from high glycosylation of the yeast-expressed RBDs, and not from aggregation. Further evidence for the glycosylation of yeast-expressed RBDs was confirmed when the N-linked glycosylation sites were deleted or mutated (N1, N2 and N3); both the extent of glycosylation and the apparent M.W. of recombinant RBDs were reduced (FIGS. 2A and 2B), accordingly. These constructs with the deglycosylated forms allowed accurate and reproducible control during scale-up production and quality control testing. Notably, expression yield was successively decreased of recombinant RBD also observed as N-linked glycosylation sites were deleted/mutated (yield level: WT>N1>N2>N3; FIGS. 2A and 2B). This suggested that a workable balance between expression yield and glycosylation level should be considered during product development.

### Example 3

### Optimization of Expression Condition

### pH and Detergent

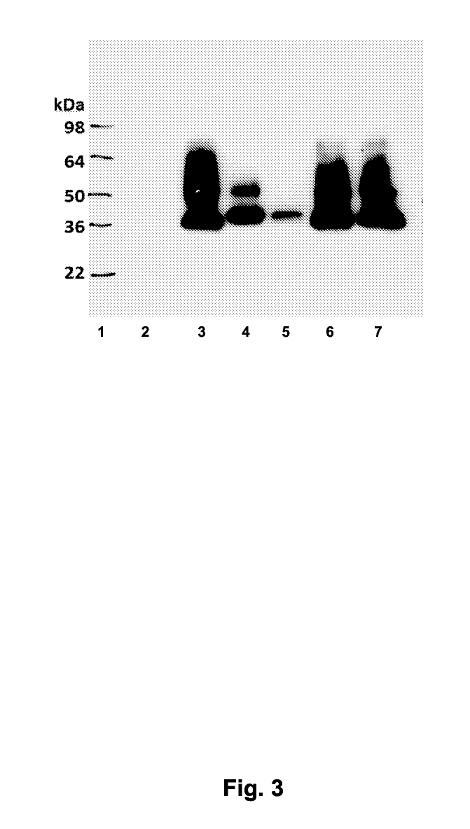

To maximize the expression yield and minimize the possible aggregation of recombinant RBDs in P. pastoris X33, RBDI93-WT was used as the prototype to test the optimized induction conditions using media with different pH and/or different concentration of Empigen detergent. Based on Western blot with anti-RBD mAb 33G4, the optimal pH for RBD193 expression was pH 6.0. No target protein was expressed in culture with pH 5.0, and even less expression of RBD193 was seen in media with pH over 6.0. The addition of (0.01% or 0.05%) Empigen detergent did not improve the expression yield or change the pattern of expressed RBD, indicating that aggregation does not affect expressed RBD193-WT (FIG. 3).

### Example 4

### Fermentation and Purification of RBD Constructs

The RBD193-WT yeast construct stopped growing when methanol induction exceeded 48 h, as a possible result of the toxicity of expressed RBD193-WT to the yeast cells. Consequently, four other constructs, including different deglycosylated forms (RBD193-N1, RBD193-N3, RBD219-WT, and RBD219-N1), were chosen for the 5 L scale fermentation in order to obtain recombinant proteins for comparison of immunogenicity and potency. After 5 L fermentation, recombinant RBD193-N1, RBD219-WT and RBD219-N1 were purified from the fermented culture by Butyl HP and size exclusion chromatography (SEC), and recombinant RBD193-N3 was purified by negative capture on Q Sepharose XL followed by SEC.

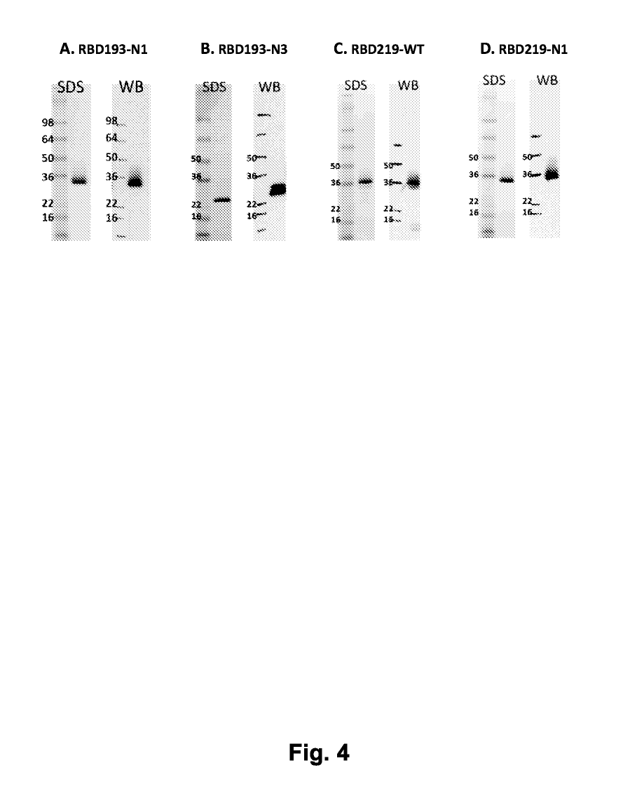

Initial attempts to purify RBD193-N1, RBD219-WT, and RBD219-N1 from the fermented culture using hydrophobic interaction (Butyl HP) chromatography and subsequent size exclusion column resulted with 95% of purified protein, as evidenced by both SDS-PAGE Coomassie-stained gels and Western blot with anti-RBD mAb 33G4 (FIG. 4). Moreover the highly glycosylated RBD and host protein contaminants could be efficiently removed with this two-step purification procedure, resulting with reliably pure and soluble products. Unlike the other expressed RBD proteins using other expression systems (Du et al., 2009; Du et al., 2010; Du et al., 2009), these yeast-expressed RBDs do not contain any 6×His tag or other tag fusion protein, thus permitting future scale-up manufacture for human use.

### Example 5

### Antigenicity Analysis of Yeast-Expressed Rbds of Sars-Cov

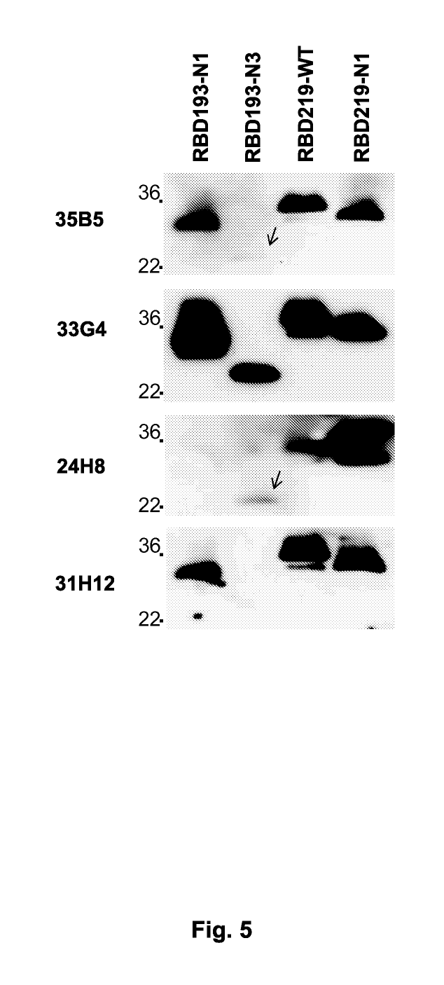

In order to confirm if the selected purified RBD constructs (RBD193-N1, RBD193-N3, RBD219-WT and RBD219-N1) shared the same antigenicity or antigenic epitopes with the mammalian cell (293T) expressed-RBD that is known can elicit the production of neutralizing antibody (Du et al., 2009; Du et al., 2010), Western blot was performed against four conformational anti-RBD mAbs, including 24H8 (Conf I), 31H12 (Conf II), 35B5 (Conf IV), and 33G4 (Conf V) (FIG. 5). Notably, mAb 33G4 strongly recognized all the RBD constructs, similarly to the recognition pattern with the mammalian cell-expressed RBD, while 24H8 recognized them only weakly. Furthermore, all yeast-expressed RBD219 constructs could be recognized more strongly by the four specific mAbs when the film exposure time was increased (data not shown), in contrast to the RBD193 constructs (Du et al., 2009; Du et al., 2010) and in agreement with previous results. This line of evidence strongly suggests that the wild-type and deglycosylated constructs exhibit similar antigenicity.

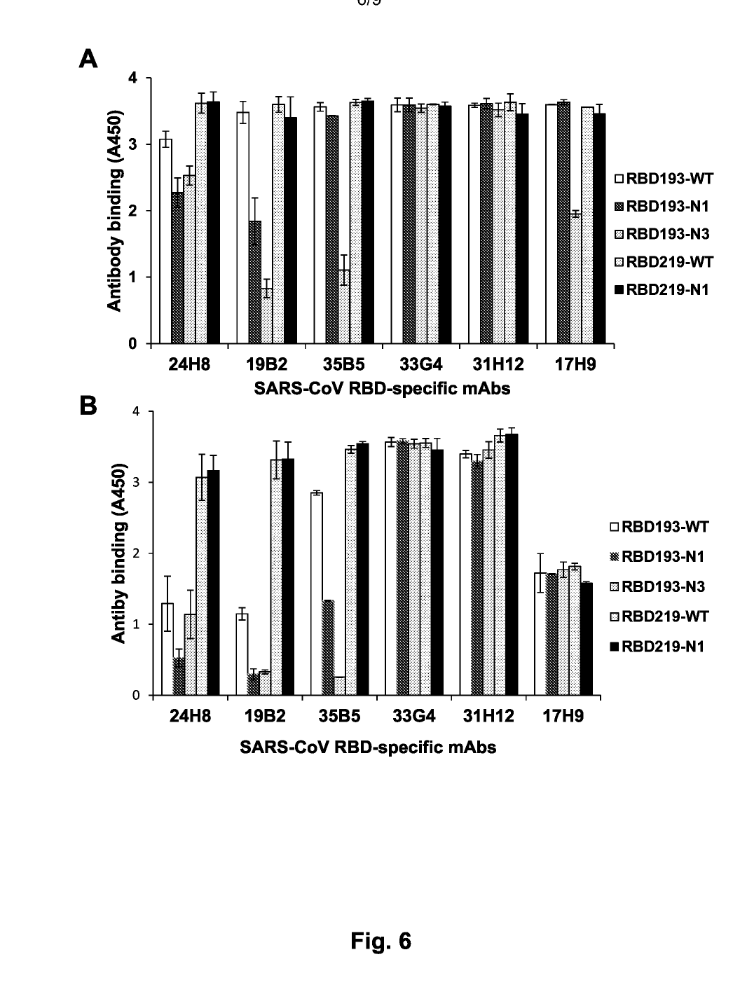

To further validate the antigenicity of these RBD proteins, ELISA was performed with five conformational anti-RBD mAbs (24H8, 31H12, 35B5, 33G4, and 19B2 (Conf VI)) and one linear anti-RBD mAb (17H9) (Goud et al., 2004) As shown in FIG. 6A, while all yeast-expressed RBDs (1 g/ml) reacted with the anti-RBD mAbs (2.2 g/ml), RBD219-WT and RBD219-N1 exhibited the strongest binding to all tested mAbs. The reactivity of RBD193-N3 with Conf IV mAb 35B5, Conf VI mAb 19B2, and linear mAb 17H9 was considerably lower than that of the other wild-type and mutant RBDs. Decrease in mAb concentration to 0.25 lag/ml did not significantly affect the binding of RBD219-N1 or RBD219-WT to all five conformational mAbs, whereas their reactivity to the linear anti-RBD mAb 17H9 was significantly decreased (FIG. 6B). These data suggest that the deglycosylated RBD219-N1 protein, like RBD219-WT, was able to maintain conformation and antigenicity, despite the deletion of the N1 glycosylation site.

### Example 6

### Functionality of Yeast-Expressed RBD Proteins

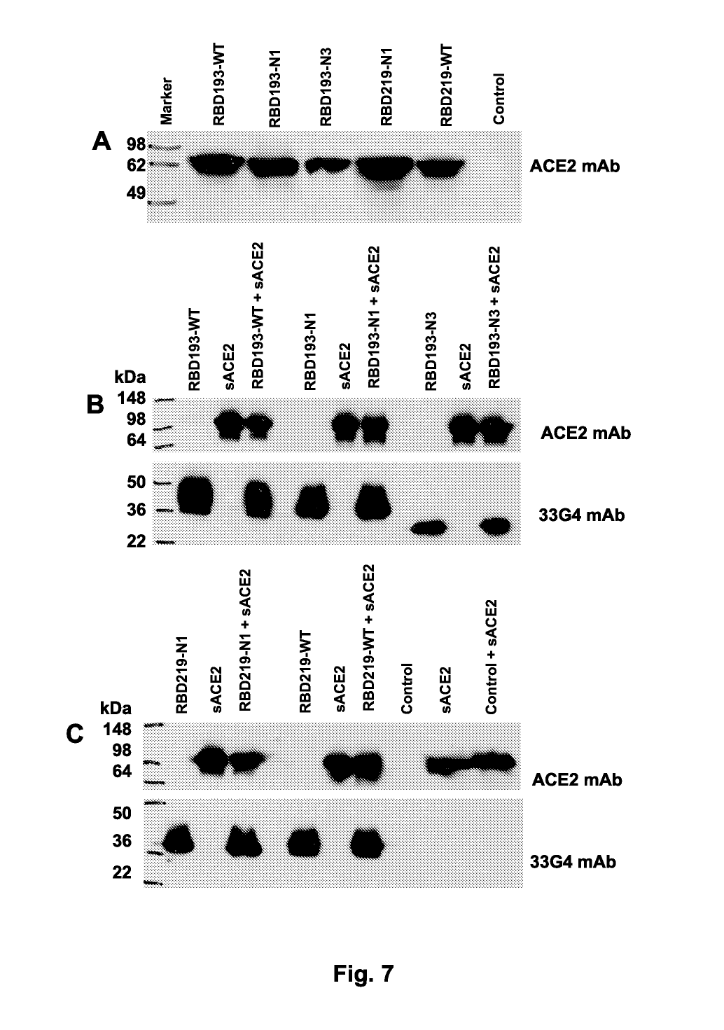

The functionality of yeast-expressed RBD proteins was further confirmed based on the ability of these proteins to bind to ACE2, the receptor of SARS-CoV, in either cell-associated or soluble form (sACE2). To establish the binding of these proteins to the cell-associated ACE2, a test protein (20 μg/each) was first mixed with ACE2/293T cells in the presence of linear anti-RBD mAb 17H9 (Goud et al., 2004) and protein A & G beads, followed by Western blot using a specific antibody against ACE2. As shown in FIG. 7A, one clear band corresponding to the size of ACE2 was observed in all cell lysates pulled down with the respective RBD proteins; all reacted strongly with the ACE2-specific mAb, although the RBD193-N3 mixture showed weaker reaction. These results suggest that the yeast-expressed RBD proteins are able to bind efficiently to the cell-surface receptor ACE2. As expected, no band (binding) was shown in the line of control protein (MERS-CoV RBD) (FIG. 7A).

The binding of the RBD proteins was further detected with sACE2 by mixing equal concentration of an RBD protein with sACE2 (containing 6×His tag) in the presence of Ni-NTA beads, followed by Western blot as above. As shown in FIGS. 7B-C, two clear bands corresponding to the size of sACE2 and respective RBD proteins were revealed in the pulled-down samples, while only one band corresponding to the size of sACE2 or respective RBD proteins, was shown in the samples containing either sACE2 or RBD protein only, all of which reacted strongly with antibodies against ACE2 or SARS-CoV RBD (33G4), respectively. However, only one band corresponding to the size of sACE2 was shown in the control containing RBD protein of MERS-CoV plus sACE2, or sACE2 only (FIG. 7B-C). These results confirm the specific binding of these yeast-expressed RBDs with SARS-CoV's receptor ACE2, indicating that all RBD proteins with or without mutations maintain sufficient functionality.

### Example 7

### Yeast-Expressed RBD Proteins of SARS-CoV Elicited a Robust Systemic Humoral Immune Response

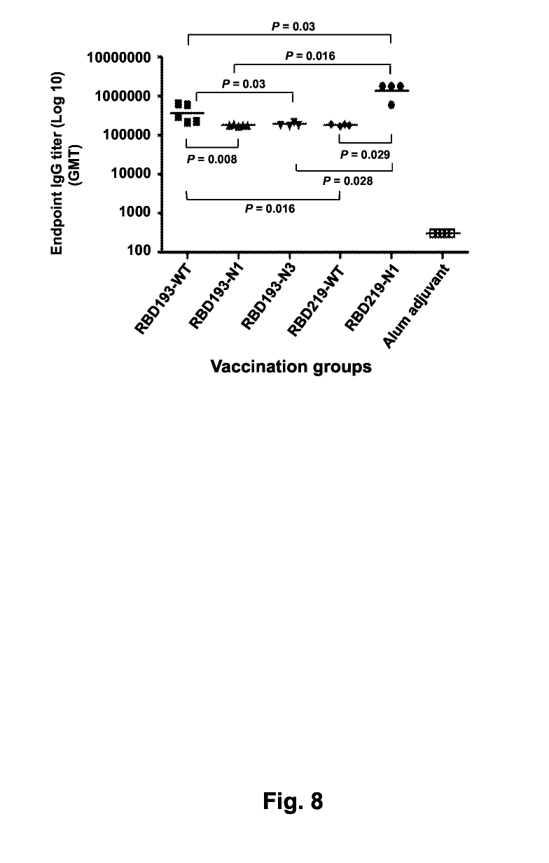

To compare the immunogenicity of yeast-expressed RBD proteins, mice were immunized using these proteins and analyzed the IgG antibody responses in mouse sera collected at 10 days post-last vaccination. As shown in FIG. 8, all yeast-expressed RBD proteins were able to induce strong IgG antibody responses against RBD219-WT. Particularly, RBD219-N1 induced significantly higher IgG antibody responses against RBD219-WT than other RBD proteins, including RBD193-WT, RBD193-N1, RBD193-N3 and RBD219-WT, with the geometric mean titers of 1.4×10⁶ (RBD219-N1), 3.5×10⁵ (RBD193-WT), 1.8×10⁵ (RBD193-N1), 1.9×10⁵ (RBD193-N3), and 1.8×10⁵ (RBD219-WT), respectively. By comparison, SARS-CoV RBD193-WT induced significantly higher IgG antibody responses against RBD219-WT than RBD193-N1, RBD193-N3 and RBD219-WT, respectively. No significant differences were observed between SARS-CoV RBD193-N1, RBD193-N3 or RBD219-WT. The control mice, which were immunized with alum plus PBS, only showed background antibody responses (FIG. 8). These results indicate that RBD219-N1 exhibited the highest immunogenicity, eliciting, in turn, the strongest RBD-specific antibody responses in the vaccinated mice.

### Example 8

### Yeast-Expressed SARS-CoV Rbd Proteins Induced in Vaccinated Mice Comparable Titers of Neutralizing Antibody Against the SARS-CoV

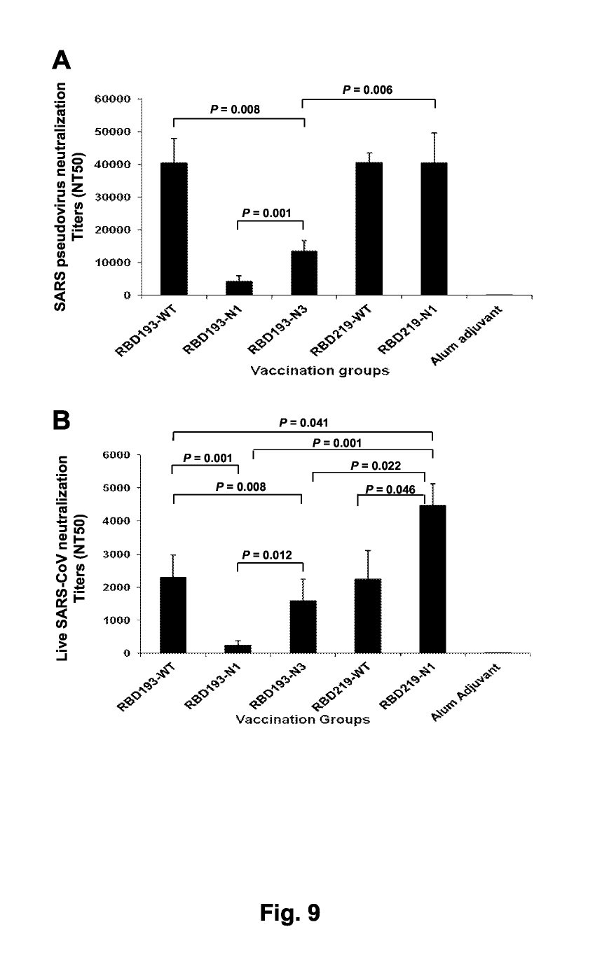

To compare the ability of the yeast-expressed SARS-CoV RBD proteins to elicit neutralizing antibodies, the sera collected from vaccinated mice 10 days after the last vaccination was tested using a SARS pseudovirus-based neutralization assay. As shown in FIG. 9A, immunization with RBD193-WT, RBD219-WT, or RBD219-N1 uniformly elicited potent neutralizing antibody responses, with the neutralizing antibody titers around 4×10⁴, respectively, which were significantly stronger than those induced by either RBD193-N1 or RBD193-N3, with the neutralizing antibody titers of 4.3×10³ and 1.4×10⁴, respectively. As expected, the alum adjuvant plus PBS control induced no neutralizing antibody response against the SARS pseudovirus (FIG. 9A).

To further confirm the functionality of the induced neutralizing antibodies, a live SARS-CoV-based neutralization assay was then performed. As shown in FIG. 9B, immunization with RBD219-N1 resulted in significantly higher titers of neutralizing antibody responses against live SARS-CoV infection than those elicited by RBD193-WT, RBD193-N1, RBD193-N3, or even RBD219-WT, with the neutralizing antibody titers reaching 4.5×10³ (RBD219-N1), 2.3×10³ (RBD193-WT), 2.5×10² (RBD193-N1), 1.6×10³ (RBD193-N3), and 2.2×10³ (RBD219-WT), respectively. While no significant difference was seen between RBD193-WT and RBD219-WT groups, the titers of neutralizing antibodies against live SARS-CoV infection induced by RBD193-N1 and RBD193-N3 were significantly lower than those induced by RBD193-WT, suggesting that the deletion of the N1 glycosylation site (RBD193-N1) and/or mutation of the 2nd and 3rd glycosylation sites (RBD193-N3) in RBD193 may have affected the conformation of neutralizing epitope in the yeast-expressed RBD193 protein. Similarly, the alum control group did not induce neutralizing antibodies against live SARS-CoV (FIG. 9B). The above data further confirm that RBD219-N1 possessed the strongest immunogenicity amongst RBD proteins tested in inducing potent neutralizing antibody responses against live SARS-CoV in vaccinated animals.

### Example 9

### Significance of Certain Embodiments

The RBD of SARS-CoV S protein contains multiple conformation-dependent epitopes that induce potent neutralizing antibody responses against a broad spectrum of SARS-CoV strains, thus serving as an important target for developing SARS vaccines (Goud et al., 2004; He et al., 2006; Punt et al., 2002; Dean, 1999). It has been previously demonstrated that the recombinant protein containing 193 amino acids of the RBD domain fused with an Fc tag (RBD193-Fc) induced highly potent neutralizing antibody responses and protective immunity in vaccinated animals (Du et al., 2009; Du et al., 2010 Du et al., 2009). To eliminate potential adverse effects in humans, possibly induced by the Fc fragment in these vaccine candidates, several RBD proteins were successively expressed without the Fc tag in different expression systems, including mammalian 293T and CHO-K1 cells, Sf9 inset cells and E. coli. It was shown that most of these RBD proteins without the Fc tag could still induce strong neutralizing antibody response and protection against SARS-CoV challenge in the vaccinated animals (Du et al., 2009; Du et al., 2010 Du et al., 2009). These findings indicate that the RBD protein on itself can elicit potent neutralizing antibody response and protect animals and in specific embodiments also people from SARS-CoV infection.

SARS-CoV S-RBD (residues 318-510) contains three N-linked glycosylation sites at the N-1, N-13 and N-40 positions (Wong et al., 2004). While a mammalian cell-expressed RBD protein with high glycosylation induced significant neutralizing activity, an E. coli-expressed RBD protein without the glycans was able to react with the RBD-specific, conformation-dependent mAbs, and to also induce substantial neutralizing antibody responses in the vaccinated animals (Du et al., 2009), suggesting that recombinant RBD proteins without the glycans can still maintain their essential antigenicity and immunogenicity when used as a candidate vaccine.

The methylotrophic yeast Pichia pastoris has been widely used for heterogeneous protein expression for pharmaceutical and vaccine applications owing to its ability to: 1) produce large amounts of protein in defined media absent animal-derived growth factors; and 2) offer easy scale-up at low cost (He et al., 2005; Shibata et al., 1985). As an eukaryotic expression system, yeast is capable of many post-translational modifications, such as proteolytic processing, folding, disulfide bond formation and glycosylation, which may be necessary for the functions of the expressed proteins. However, different from mammalian cells, Pichia pastoris frequently adds monosaccharide to expressed protein to become hyperglycosylated (Hopkins et al., 2011). The extent and linkage of residues present in hyperglycosylated glycans vary, depending on yeast strain and cell culture conditions (Li et al., 2005), causing, as a consequence, concerns regarding expression yield, reproducibility of homogeneous products and quality control (Prabakaran et al., 2006). Because of these concerns, the three asparagines responsible for N-glycosylation in the RBD sequence were removed or mutated to make deglycosylated RBD forms.

In the present disclosure, two wild-type RBDs were expressed with different lengths (RBD193-WT and RBD219-WT) and their deglycosylated mutants (RBDI93-N1, RBD193-N2, RBD193-N3; RBD219-N1, RBD219-N2, RBD219-N3) by mutating or deleting residues at the corresponding glycosylation sites (FIG. 1). Their antigenicity, functionality and immunogenicity were then compared. SDS-PAGE analysis revealed that both yeast-expressed RBDI93-WT and RBD219-WT migrated as a smear with apparent molecular weights higher than their calculated M.W., suggesting that these recombinant RBD-WTs were highly glycosylated or aggregated (FIG. 2B). After digestion of RBDI93-WT with N-glycosidase PNGase F, the high M.W. smear disappeared, and the size of RBD193-WT returned to the expected M.W. (FIG. 2C), confirming that the high M.W. smear was from overglycosylation of the yeast-expressed RBD, rather than any aggregation. When some of the N-linked glycosylation sites in RBD193-WT and RBD219-WT were deleted or mutated (N1, N2 and N3), the apparent M.W. of recombinant RBDs was reduced accordingly (FIG. 2B). Therefore, these deglycosylated mutants have allowed us to accurately and reproducibly control the expression process during scale-up production and quality control testing. Notably, the expression yield of the recombinant RBDs with deleted or mutated N-linked glycosylation sites was reduced compared to that of their corresponding wild-type RBDs (FIG. 2B); therefore, pointing to the need to find a workable balance between expression yield and glycosylation level during product development.

Importantly, RBD219-N1 exhibited a lower glycosylation level without obvious compromised expression yield when compared to the wild-type form (FIG. 2B). After two-step purification, including hydrophobic interaction (Butyl HP) chromatography and size exclusion column, most of the glycosylation species and the host protein contaminations were removed (FIGS. 3 and 4). Consequently, the recombinant RBD219-N1 protein was selected for further evaluation. RBD219-N1 was highly recognized by the conformational anti-RBD mAbs (Goud et al., 2004; Dean, 1999) using two separate methods; Western blot (FIG. 5), and ELISA (FIG. 6), further validating the ability of this deglycosylated protein to maintain antigenicity that is equal to that of the wild-type protein. Moreover, like the wild-type RBD, RBD219-N1 bound very well to the cell-associated and soluble receptor ACE2 (FIG. 7), further confirming that this yeast-expressed RBD mutant maintains its functionality.

The immunogenicity was compared of RBD219-N1 with wild-type RBDs and some of the other deglycosylated proteins, it appeared that RBD219-N1 induced remarkably higher SARS-CoV RBD-specific IgG antibody responses than the wild-type RBDs, RBD219-WT and RBD193-WT, and the other deglycosylated proteins, such as RBD193-N1 and RBD193-N3; RBD193-N1 and RBD193-N3 exhibited relatively lower antigenicity and immunogenicity than RBD193-WT (FIGS. 6, 8 and 9). Among all the tested yeast-expressed RBDs, RBD219-N1 elicited the highest titers of neutralizing antibodies against the pseudotyped SARS-CoV and live SARS-CoV infection (FIG. 9). In specific embodiments, deletion of the N-1 glycosylation site in RBD219-N1 may have unmasked a neutralizing epitope in RBD, leading to better induction of neutralizing antibody responses. A similar phenomenon was observed in Ebola virus. Mutation of two N-linked sites on the Ebola virus glycoprotein subunit 1 (GP1) resulted in enhanced immunogenicity, possibly by unmasking the protective antibody epitopes on GP1 (Zakhartchouk et al., 2007).

Further evaluation of the RBD219-N1 vaccine candidate in vivo for efficacy, protecting animals from SARS-CoV challenge, is performed. According to previous experience, an RBD-based vaccine candidate that can induce NT50>1,000 of neutralizing antibody titers against the live SARS-CoV is expected to fully protect animals from infection by the virus (Du et al., 2009; Du et al., 2009). The present data showed that the titer of neutralizing antibodies induced by RBD219-N1 in vaccinated mice against live SARS-CoV was >2,000 (FIG. 9B), demonstrating its prospect to efficiently protect immunized animals from an SARS-CoV challenge.

In conclusion, the yeast-expressed RBD without any extra tag but with one deleted glycosylation site, RBD219-N1, can exhibit lower glycosylation levels and higher expression yield, and induced stronger RBD-specific antibody responses and more potent neutralizing antibodies against both pseudotyped and live SARS-CoV, pointing to its use for as an effective and safe subunit vaccine against SARS-CoV in humans.

### Example 10

### Example of Fermentation Processes

Examples of procedures for upstream and downstream processing of an example of a RBD composition from SARS-CoV spike protein are described as follows:

Upstream Process—Fermentation:

A 500 mL culture in BMG was inoculated with the clone RBD219-N1/pPICZaA/P. pastoris X33 and incubated at 30° C., 225 rpm for 18-24 hours.

After that, the overnight culture was used to inoculate the medium in the fermenter. Fermentation was initiated at 30° C. and the initial pH set at 5.0. Gas and agitation was adjusted to maintain DO at 30%. Upon exhaustion of glycerol during the batch phase (DO spike), the culture was starved for one hour (no feed). During the one hour starvation, the temperature was shifted from 30° C. to 25° C. and the pH was increased from 5.0 to 6.5. After the pH and temperature ramp, methanol induction was initiated. For the first 6 hours of induction (hour 0 to hour 6), the methanol feed rate was ramped from 1 ml/L/hr to 11 ml/L/hr. After the 6 hour ramp, the methanol feed rate was maintained at 11 ml/L/hr for 18 hours (hour 6 to hour 24), after an 18 hour of steady flow rate at 11 mL/L/hr, the methanol flow rate was ramped again from 11 to 13 mL/L/hr over 6 hours (hour 24 to hour 30) and was maintained at 13 mL/L/hr for another 18 hours (hour 30 to hour 48); finally, after 18 hour of steady flow rate at 13 mL/L/hr, the flow rate ramped from 13-15 mL/L/hr over 6 hours (from hour 48 to hour 54) and maintained at 15 mL/L/hr for the remainder of the run.

After fermentation, the culture was harvested by aseptically pumping the culture into a sterile harvest container. Cells were removed by centrifugation at 7000 rpm (˜12,500×g) for 30 minutes at 4° C. using a JLA 8.1000 rotor. The supernatant was filtered using 0.22 um bottle-top filtration units and the filtered supernatant was transferred to 1 L PETG bottles and was stored until purification.

Downstream Process—Purification:

The fermentation supernatant was concentrated 3-4 folds. Ammonium hydroxide and Ammonium sulfate were added in the concentrated fermentation supernatant to adjust its pH and conductivity to around 8.0±5 and 220±10 ms/cm respectively.

After the pH and conductivity adjustment, the concentrated fermentation supernatant was filtered through 0.22 um and loaded onto the Butyl HP column with binding condition of 30 mM Tris, 2M Ammonium sulfate (AS) at pH 8.0. The column was washed with 2 column volume (CV) of 30 mM Tris, 2M AS at pH8.0 to remove the unbound protein in the column and followed by 10 V of the first step elution with 30 mM Tris, 0.7M AS at pH 8.0 to remove the bound contaminants, finally a 10 CV of the second step elution with 30 mM Tris at pH 8.0 to obtain the elution pool.

The elution pool was further concentrated 40±10 fold and loaded onto Superdex75 size exclusion column to remove the rest of the contaminants.

In order to characterize RBD219-N1 and to establish the chemical stability, one or more protein-specific assays may be utilized:

| Assay name | Purpose |
|---|---|
| pH and appearance | Identity and stability |
| Endotoxin test | Analysis of contamination |
| SDS-PAGE (reduced and non-reduced) | Identity, purity and stability |
| Western Blot | Identity, purity and stability |
| Host cell protein blot | Purity |
| HPLC-SEC (along with light scattering and | Identity, purity and stability |
| refractive index) | |
| UPLC-RP | Identity, finger printing, |
| | quality and stability |

Other assay

| | |
|---|---|
| UPLC-HILIC | Glycan analysis |

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### PUBLICATIONS

Du L, He Y, Zhou Y, Liu S, Zheng B J, Jiang S. The spike protein of SARS-CoV: a target for vaccine and therapeutic development. Nat. Rev. Microbiol. 2009; 7(3):226-36.
Peiris J S M, Lai S T, Poon L L M, et al. Coronavirus as a possible cause of severe acute respiratory syndrome. Lancet 2003; 361(9366):1319-25.
Zhong N S, Zheng B J, Li Y M, et al. Epidemiology and cause of severe acute respiratory syndrome (SARS) in Guangdong, People's Republic of China, in February, 2003. Lancet 2003; 362(9393): 1353-8.
Jiang S, Bottazzi M E, Du L, et al. Roadmap to developing a recombinant coronavirus S protein receptor-binding domain vaccine for severe acute respiratory syndrome. Expert Rev. Vaccines 2012; 11(12):1405-13.
Perlman S, Dandekar A A. Immunopathogenesis of coronavirus infections: implications for SARS. Nat Rev Immunol. 2005; 5(12):917-27.
Bolles M, Deming D, Long K, et al. A double-inactivated SARS-CoV vaccine provides incomplete protection in mice and induces increased eosinophilic pro-inflammatory pulmonary response upon challenge. J. Virol. 2011; 85(23):12201-15.(23):12201-15.
Castilow E M, Olson M R, Varga S M. Understanding respiratory syncytial virus (RSV) vaccine-enhanced disease. Immunol. Res. 2007; 39(1-3):225-39.
Tseng C T, Sbrana E, Iwata-Yoshikawa N. et al. Immunization with SARS coronavirus vaccines leads to pulmonary immunopathology on challenge with the SARS virus. PLoS ONE 2012; 7(4):e35421
Jaume M, Yip M S, Kam Y W, et al. SARS CoV subunit vaccine: antibody-mediated neutralisation and enhancement. Hong. Kong. Med J 2012; 18 Suppl 2:31-6.
Wong S K, Li W H, Moore M J, Choe H, Farzan M. A 193-amino acid fragment of the SARS coronavirus S protein efficiently binds angiotensin-converting enzyme 2. J. Biol. Chem. 2004; 279(5):3197-201.
Du L, Zhao G, Chan C C, et al. Recombinant receptor-binding domain of SARS-CoV spike protein expressed in mammalian, insect and E. coli cells elicits potent neutralizing antibody and protective immunity. Virology 2009; 393(1):144-50.
Du L, Zhao G, Chan C C, et al. A 219-mer CHO-expressing receptor-binding domain of SARS-CoV S protein induces potent immune responses and protective immunity. Viral Immunol 2010; 23(2):211-9.
Chen Z, Zhang L, Qin C, et al. Recombinant modified vaccinia virus Ankara expressing the spike glycoprotein of severe acute respiratory syndrome coronavirus induces protective neutralizing antibodies primarily targeting the receptor binding region. J. Virol. 2005; 79(5):2678-88.
Goud G N, Zhan B, Ghosh K, et al. Cloning, yeast expression, isolation, and vaccine testing of recombinant Ancylostoma-secreted protein (ASP)-1 and ASP-2 from Ancylostoma ceylanicum. J. Infect. Dis. 2004; 189(5):919-29.
Du L, Zhao G, Li L, et al. Antigenicity and immunogenicity of SARS-CoV S protein receptor-binding domain stably expressed in CHO cells. Biochem. Biophys. Res. Commun. 2009; 384(4):486-90.
He Y, Lu H, Siddiqui P, Zhou Y, Jiang S. Receptor-binding domain of severe acute respiratory syndrome coronavirus spike protein contains multiple conformation-dependent epitopes that induce highly potent neutralizing antibodies. J Immunol. 2005; 174(8):4908-15.
Du L, Zhao G, Kou Z, et al. Identification of a receptor-binding domain in the s protein of the novel human coronavirus middle East respiratory syndrome coronavirus as an essential target for vaccine development. J. Virol. 2013; 87(17):9939-42.
Du L, Zhao G, Kou Z, et al. Identification of a receptor-binding domain in the S protein of the novel human coronavirus Middle East respiratory syndrome coronavirus as an essential target for vaccine development. J. Virol. 2013; 87(17):9939-42.
Du L, Zhao G, Kou Z, et al. Identification of a receptor-binding domain in the s protein of the novel human coronavirus middle East respiratory syndrome coronavirus as an essential target for vaccine development. J. Virol. 2013; 87(17):9939-42.
Du L, Zhao G, Sun S, et al. A critical HA1 neutralizing domain of H5N1 influenza in an optimal conformation induces strong cross-protection. PLoS ONE 2013; 8(1):e53568
Du L, Leung V H, Zhang X, et al. A recombinant vaccine of H5N1 HA1 fused with foldon and human IgG Fc induced complete cross-clade protection against divergent H5N1 viruses. PLoS. ONE. 2011; 6(1):e16555
Du L, Zhao G, Lin Y, et al. Intranasal vaccination of recombinant adeno-associated virus encoding receptor-binding domain of severe acute respiratory syndrome coronavirus (SARS-CoV) spike protein induces strong mucosal immune responses and provides long-term protection against SARS-CoV infection. J. Immunol. 2008; 180(2):948-56.
Du L, Zhao G. Lin Y, et al. Priming with rAAV encoding RBD of SARS-CoV S protein and boosting with RBD-specific peptides for T cell epitopes elevated humoral and cellular immune responses against SARS-CoV infection. Vaccine 2008; 26(13):1644-51.
He Y, Zhou Y, Liu S, et al. Receptor-binding domain of SARS-CoV spike protein induces highly potent neutralizing antibodies: implication for developing subunit vaccine. Biochem. Biophys. Res. Commun. 2004; 324(2):773-81.
He Y, Zhu Q, Liu S, et al. Identification of a critical neutralization determinant of severe acute respiratory syndrome (SARS)-associated coronavirus: Importance for designing SARS vaccines. Virology 2005; 334(1):74-82.
He Y, Li J, Li W, Lustigman S, Farzan M, Jiang S. Cross-neutralization of human and palm civet severe acute respiratory syndrome coronaviruses by antibodies targeting the receptor-binding domain of spike protein. J. Immunol. 2006; 176(10):6085-92.
Punt P J, van B N, Conesa A, Albers A, Mangnus J, van den H C. Filamentous fungi as cell factories for heterologous protein production. Trends Biotechnol. 2002; 20(5):200-6.
Dean N. Asparagine-linked glycosylation in the yeast Golgi. Biochim. Biophys. Acta 1999; 1426(2):309-22.
Shibata N, Ichikawa T, Tojo M, et al. Immunochemical study on the mannans of Candida albicans NIH A-207, NIH B-792, and J-1012 strains prepared by fractional precipitation with cetyltrimethylammonium bromide. Arch. Biochem. Biophys. 1985; 243(2):338-48.
Hopkins D, Gomathinayagam S, Rittenhour A M, et al. Elimination of beta-mannose glycan structures in Pichia pastoris. Glycobiology 2011; 21(12):1616-26.
Dowling W, Thompson E, Badger C, et al. Influences of glycosylation on antigenicity, immunogenicity, and protective efficacy of ebola virus G P DNA vaccines. J. Virol. 2007; 81(4):1821-37.

1. An isolated composition comprising the receptor-binding domain (RBD) of the Severe acute respiratory syndrome coronavirus (SARS-CoV) spike protein, wherein said domain lacks at least one glycosylation site or is deglycosylated at least at one site that is glycosylated under normal conditions.

2. The composition of claim 1, wherein the domain is comprised within the full-length SARS CoV spike protein.

3. The composition of claim 1, wherein the domain is a fragment of the SARS-CoV spike protein.

4. The composition of claim 3, wherein the fragment comprises amino acid residues 318-510 of the SARS CoV spike protein.

5. The composition of claim 3, wherein the fragment comprises amino acid residues 318-536 of the SARS CoV spike protein.

6. The composition of claim 3, wherein the fragment is at least 190 amino acids in length.

7. The composition of claim 3, wherein the fragment is at least 210 amino acids in length.

8. The composition of claim 1, wherein the glycosylation site is an N-glycosylation site.

9. The composition of claim 8, wherein the N-glycosylation site is an asparagine site.

10. The composition of claim 1, wherein the site is at the asparagine at amino acid 318 of the SARS-CoV spike protein.

11. The composition of claim 1, wherein the site is at the asparagine at amino acid 330 of the SARS-CoV spike protein.

12. The composition of claim 1, wherein the site is at the asparagine at amino acid 347 of the SARS-CoV spike protein.

13. The composition of claim 1, wherein the site is at one or more asparagines selected from the group consisting of amino acid 318, amino acid 330, amino acid 347 and a combination thereof of the SARS-CoV spike protein.

14. The composition of claim 1, wherein the fragment comprises amino acid residues 318-536 of the SARS CoV spike protein and the site is at the asparagine at amino acid 318 of the SARS-CoV spike protein.

15. The composition of claim 1, wherein the site comprises an amino acid deletion.

16. The composition of claim 1, wherein the site comprises an amino acid substitution.

17. The composition of claim 15, wherein the amino acid substitution is to a serine or alanine.

18. The composition of claim 1, comprised in a pharmaceutically acceptable vehicle.

19. A method of preventing or delaying the onset of SARS or of reducing at least one symptom of SARS in an individual, comprising the step of providing an effective amount of the composition of claim 1 to the individual.

20. The method of claim 18, wherein the composition is provided to the individual once.

21. The method of claim 18, wherein the composition is provided to the individual more than once.

22. The method of claim 18, wherein the composition is provided subsequently to the individual within weeks, months, or years of the first providing step.

23. The method of claim 18, wherein the individual displays one or more symptoms of SARS.

24. The method of claim 18, wherein the individual lacks any symptoms of SARS.

25. The method of claim 18, wherein the individual has been exposed to SARS.

26. The method of claim 18, wherein the individual has come into contact with an individual that has SARS.

27. The method of claim 18, wherein the individual is a child, an elderly person, exposed to a bioweapon or at risk thereof, is a member of the military, or is a health care worker.


SEQUENCE LISTING (4 sequences)

SEQ ID NO: 1 - DNA, 582 bp, Unknown
  SARS coronavirus
aacatcaccaacctgtgccccttcggcgaggtgttcaacgccaccaagttccccagcgtatacgcctgggagaggaagaagatcagcaactgcgtggccgactacagcgtgctgtacaacagcaccttcttcagcaccttcaagtgctacggcgtgagcgccaccaagctgaacgacctgtgcttcagcaacgtgtacgccgacagcttcgtggtgaagggcgacgacgtgaggcagatcgctcctggacagaccggcgtgatcgccgactacaactacaagctgcccgacgacttcatgggctgcgtgctggcctggaacaccaggaacatcgacgccaccagcaccggcaactacaactacaagtacaggtacctgaggcacggcaagctgaggcccttcgagagggacatcagcaacgtgccattcagccctgacggcaagccctgcacaccacctgccctgaactgctactggccactgaacgactacggcttctacaccaccaccggcatcggctaccagccctacagggtggtggtgctgagcttcgagctgctgaacgctcctgccaccgtgtga

SEQ ID NO: 2 - protein, 193 aa, Unknown
  SARS coronavirus
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV

SEQ ID NO: 3 - DNA, 657 bp, Unknown
  SARS coronavirus
aacatcaccaacctgtgccccttcggcgaggtgttcaacgccaccaagttccccagcgtatacgcctgggagaggaagaagatcagcaactgcgtggccgactacagcgtgctgtacaacagcaccttcttcagcaccttcaagtgctacggcgtgagcgccaccaagctgaacgacctgtgcttcagcaacgtgtacgccgacagcttcgtggtgaagggcgacgacgtgaggcagatcgctcctggacagaccggcgtgatcgccgactacaactacaagctgcccgacgacttcatgggctgcgtgctggcctggaacaccaggaacatcgacgccaccagcaccggcaactacaactacaagtacaggtacctgaggcacggcaagctgaggcccttcgagagggacatcagcaacgtgccattcagccctgacggcaagccctgcacaccacctgccctgaactgctactggccactgaacgactacggcttctacaccaccaccggcatcggctaccagccctacagggtggtggtgctgagcttcgagctgctgaacgctcctgccaccgtgtgcggccctaagctgagcaccgacctgatcaagaaccagtgcgtgaacttcaacttcaacggcctgaccggcaccggc

SEQ ID NO: 4 - protein, 219 aa, Unknown
  SARS coronavirus
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTG